(19) United States
(12) Patent Application Publication
(10) Pub. No.: US 2006/0120995 A1
(43) Pub. Date: Jun. 8, 2006

(54) Neoadjuvant genetic compositions and methods

(76) Inventor: Maulik R. Shah, St. Louis, MO (US)

Correspondence Address: SAINT LOUIS UNIVERSITY;OFFICE OF RESEARCH ADMINISTRATION, 3556 CAROLINE STREET, ST. LOUIS, MO, 63104, US

(21) Appl. No.: 11/002,839

(22) Filed: Dec. 2, 2004

(51) Int. Cl. A61K 48/00; A61K 38/19; A61K 38/17

(52) U.S. Cl. 424/85.1; 424/93.21; 514/12

(57) ABSTRACT

The present invention relates to a method and composition for the use of a neoadjuvant to be used in with existing therapies to break host immune tolerance to tumor cells or other types of diseased cells. More precisely the present invention is directed to a vaccine which delivers a polypeptide adjuvant such as HSP72, or a cytokine molecule such as GMCSF, which is encoded in a polynucleotide that will be expressed at high levels within tumor cells and may also bind to tumor cell antigens. The vaccine may be administered alone, or in conjunction with a known tumor antigen or vaccine. After a period of time sufficient for expression of the polynucleotide and for the binding of adjuvant to cancer antigens, the cancer will be treated with conventional therapies, allowing the adjuvant/antigen complex to be released to the interstitial fluids where they will be accessible to antigen presenting cells of the host immune system. The presentation of the cancer antigen complexed with adjuvant as well as cytokine stimulation of the host immune system will cause the host to mount a heightened immune response against all remaining cancer cells or tumors which share these antigens.

### BACKGROUND OF THE INVENTION

1. Field of the Invention

The invention relates generally to compositions and methods of treating a disease using an immune system adjuvant in combination with another therapy. Specifically, the invention relates to compositions and methods of using heat shock protein therapy in conjunction with antigen presentation to improve therapeutic outcome.

2. Description of the Related Art

### Neoadjuvant Therapy

Neoadjuvant therapy is a method of treating a patient by first priming and/or educating a patient's immune system against a target before the administration of a second therapy. Usually, neoadjuvant therapy is used in cancer treatment, in conjunction with chemotherapy or surgery, to help generate anti-tumor immune responses capable of impeding micrometastatic disease and/or prevention of tumor recurrence. Immunotherapy in this setting has heretofore been limited to immunochemotherapy with the use of immune system modifying biologicals such as interferon (see Quan and Palackdharry, Dis Mon; 43: 745-808, 1997), or immuno-cell therapy with the use of activated immune effector cells like dendritic cells (see Morse et al. Int J Gastrointest Cancer; 32: 1-6, 2002) or modified tumor cell vaccines (see Delman K A et al. Ann Surg; 236: 337-342, 2002). Results of animal studies and limited clinical trials suggest that neoadjuvant immunotherapy may have a role in the treatment of immunogenic cancers (Sabel M S et al. Ann Surg Oncol; 11: 147-156, 2004; Buter and Pinedo Curr Oncol Rep; 5: 171-176, 2003; and Bodar E et al. Oncology (Huntingt); 16: 32-39, 2002). These methods have a number of drawbacks related to lack of specificity for the tumor.

### Heat Shock Proteins as Immunomodulatory Agents

Heat shock proteins belong to the molecular chaperone family and are induced by various noxious stimuli including heat, from which their name is derived (Haslbeck Cell Mol Life Sci., 59(10):1649-57, (2002). Srivastava et al. demonstrated that antigenic peptides from processed tumor associated antigens were bound to heat shock protein 70 and 90 (“HSP70” and n“HSP90”, respectively) and that these proteins were responsible for conferring tumor immunity (Srivastava, Drug News Perspect. 13(9): 517-22, 2000). Subsequently, numerous studies have shown the efficacy of vaccination with tumor-derived purified HSP-antigen complexes to generate anti-tumor immune response, which are capable of conferring tumor resistance with specificity for the HSP associated tumor antigen (Castelli et al. Cancer Immunol Immunother., 53(3): 227-33, 2004; Srivastava, and Amato, Vaccine., 19(17-19): 2590-7, 2001); and Yedavelli et al., Int J Mol Med., 4(3): 243-8, 1999). Some of the proposed mechanisms for tumor immunology include an increase in tumor immunogenicity associated with HSP72 expression, promotion of antigen presentation (Melcher et al., Nat Med., 4(5): 581-7, 1998), and stimulation of antigen presenting cells to secrete proinflammatory cytokines (Somersan et al. J Immunol., 167(9): 4844-52, 2001).

HSP72 was previously identified to be a component of high molecular weight tumor cell lysate, which conferred tumor immunity in conjunction with tumor cell based vaccines (Udono and Srivastava, J Exp Med; 178: 1391-1396, 1993). HSP72, for example, has the capability of binding antigenic peptides and increasing tumor immunogenicity (Blachere, and Srivastava Semin Cancer Biol; 6: 349-355, 1995). Additionally, HSP72 has been shown to activate antigen-presenting cells to augment the generation of an immune response (Menoret and Srivastava Curr Opin Immunol; 14: 45-51, 2002).

### DNA Vaccines

DNA vaccination has previously been shown to be a safe and effective method for inducing antigen-specific cytotoxic T-cell responses and has rapidly been developed as a potential new vaccination strategy (see Ada and Ramshaw, Expert Opin Emerg Drugs., 8(1): 27-35, 2003). Although DNA vaccines have been studied for a number of pathogens and tumor models, immune responses have not been as robust as conventional vaccines.

Recombinant adenoviruses expressing antigens have been well studied as priming vaccination vectors (see Randrianarison-Jewtoukoff, Biologicals., 23(2): 145-57, 1995). Their capacity to induce inflammatory responses resulting in prolonged cellular immune responses makes them ideal vectors as immune adjuvants (Truckenmiller and Norbury, Expert Opin Biol Ther., 4(6): 861-8, 2004). Although they have been used extensively as a method of delivering an antigenic protein, no studies have utilized their potential as immune adjuvants for DNA vaccination.

### Combining Immunomodulators with DNA Vaccines

Rothman et alia have taught the use of a hybrid antigen, which contains an antigenic region and an HSP70 binding domain covalently linked together by an amino acid linker (U.S. Pat. No. 6,761,892). This hybrid polypeptide may be introduced with HSP70 polypeptide, or each may be introduced as polynucleotide in the form of a DNA vaccine. Hybrid molecules such as these were shown to invoke an immune response against those antigenic domains. However this method suffers from having to first identify the appropriate antigen present in a particular patient's tumor, and then constructing a hybrid vaccine.

Melcher et al. have shown that over expression of HSP70 in tumor cells in mice significantly increased the immunogenicity of those tumors (Melcher et al. Nature Medicine (4) δ: 581-7, 1998). Melcher first transfected tumor cells with HSP70 encoding DNA in vitro, and subsequently injected these cells subcutaneously into mice. They found when tumors grew and regressed, or were surgically removed, the mice were protected against rechallenge with cells of the same prenatal linage. Although Melcher et alia had shown that the expression of HSP70 in tumor cells in mice can be effective at breaking immunotolerance, they did not identify a practical way to deliver a HSP vaccine to a patient, nor had they provided a method that allows for optimal education or priming of the immune system.

Haviv et al. used a replication deficient adenovirus to transfect cancer cells in vitro with HSP 70 (Haviv et al. Cancer Research 61, 8361-8365, 2001). This was not done to break immunotolerance to tumors, but rather to exploit another property of HSPs, the ability to enhance the cell killing effect of a subsequent infection by a replication competent adenovirus. Although the second adenovirus resulted in destruction of the infected cells, this work was performed in vitro, and there was no attempt to apply this as a neoadjuvant therapy either in humans or in an animal model.

Huang et al. disclosed a recombinant adenovirus, which possess the dual properties of HSP over expression and oncolytic activity (Huang et al. Cancer Research 63, 7321-7329, 2003). Similarly to Haviv, the purpose was to exploit the enhanced killing effect of HSP expression within a virus vector and not for use as a neoadjuvant. Furthermore with both activities contained in one vector, there is not allowance for a therapeutically effective period of time for maximum HSP expression, and immune system education and priming.

Previous disclosures have used HSP's in combination with adenovirus, but not as a neoadjuvant in combination with a subsequent therapy. They have not incorporated an effective method of educating and priming the host immune system. The present invention teaches that effectively introducing an HSP adjuvant, followed by a therapeutically effective period of time to allow the education and priming of the immune system before further treatment of the disease, provides a maximum immunological response and protection against further development of disease.

### SUMMARY OF THE INVENTION

The inventor has made the surprising discovery that the management of a disease can be dramatically improved when the immune system of a patient is put on alert to attack a disease in advance of a second treatment to reduce the overall disease burden in the patient. While others have administered immunomodulatory agents, such as HSP72, along with a disease antigen or a cell lytic agent, e.g., adenovirus death protein to promote cell killing, the inventor is not aware of any application of an immunomodulatory agent as a neoadjuvant, injected directly into the tumor to enhance the cure rate of traditional therapies. The inventor has succeeded in developing methods and compositions to enhance the effectiveness of known treatment regimens by the administering to a patient a neoadjuvant prior to the known treatment. In a preferred embodiment, the neoadjuvant is an adenovirus vector that encodes an immunomodulatory polypeptide.

In one embodiment, the invention is drawn to a method of treating any disease, wherein an adjuvant is administered to a patient. The disease burden in the patient is then reduced through a disease treatment regimen. An adjuvant may be immunostimulatory in nature, antigen presenting in nature, or both. While not wishing to be bound by theory, the skilled artisan may reasonably assume that the adjuvant, if it is antigen presenting in nature, combines with a disease-associated antigen and helps to present that antigen to an immune system component. The antigen/adjuvant complex presented to the immune system elicits a heightened immune response, thus preventing the further development of the disease. While the invention is not limited to any particular mode of administration of adjuvant, in a preferred aspect of the invention the adjuvant is applied in direct contact to a disease cell. Also, while the inventor envisions that any and all diseases (e.g., tuberculosis, AIDS) can be treated through this invention, the preferred disease is cancer, wherein the adjuvant is injected intratumorally. More preferably, the adjuvant is encoded as a polynucleotide in a replication deficient adenovirus vector, which is injected into a tumor in a therapeutically effective amount, and after a therapeutically effective period of time passes to mallow for the appropriate stimulation of the immune system, the tumor is removed by surgery or chemotherapy. In this embodiment, the tumor cells naturally provide the tumor-specific antigens that combine with the adjuvant.

In a preferred aspect of this embodiment (supra), the cancer is a breast cancer. Breast cancer is often identified early due to methods employed for early screening. Despite adequate treatment, there is potential for relapse or there is micro-metastatic disease requiring adjuvant therapy in addition to surgery. Neoadjuvant gene therapy for breast cancer would involve treatment of patients prior to their surgery by direct gene transfer of an immunomodulatory agent to the cancer site. Before removing the primary tumor, the patient is treated with a composition consisting of a recombinant adenovirus containing an immunomodulatory molecule such as HSP72 or GMCSF.

In another embodiment, the invention is drawn to a method of treating any disease, wherein an adjuvant and an antigen are administered to a patient. This embodiment is similar to the first embodiment with the additional step of administering an antigen that is associated with a particular disease. For example, if a patient is suffering from AIDS, the disease antigen might be a HIV molecule. In a preferred aspect of this invention, the disease is a cancer and the antigen is a cancer-specific antigen, such as for example colorectal cancer and cancer-specific antigen CEA, or the cancer is melanoma and cancer-specific antigen tyrosinase or the like. In this preferred aspect, the cancer-specific antigen and adjuvant are injected into a tumor, then, after waiting a therapeutically-effective time, the tumor is treated. More preferably, the adjuvant is encoded as a polynucleotide in a replication deficient adenovirus vector. More preferably, the tumor is treated by being surgically removed.

In yet another embodiment, the invention is drawn to a method of treating any disease wherein the adjuvant is an endogenous or ectopic heat shock protein, which is induced to high levels of expression by applying a stress (e.g., heat, chemical shock, physical shock, biological agent) to the disease cell. In the preferred aspect, heat may be applied through electromagnetic radiation and concentrated on a tumor in such a way as to induce high levels of expression of HSPs. After waiting a therapeutically-effective time, the tumor is then treated or removed, and the patient mounts a heightened immune response, therefore preventing further progression of the disease. In another aspect, if the HSP is ectopically expressed on an exogenous polynucleotide operably linked to a regulatable promoter, activation of the promoter sequence may be used to induce over expression of HSP molecules prior to removable of the tumor.

In yet another embodiment the invention is drawn to a composition comprising a neoadjuvant, which can enhance the immune response of a patient. Preferably, the composition comprises an adenovirus comprising a first polynucleotide encoding a polypeptide adjuvant and a second polynucleotide encoding an immunomodulatory agent, such that, when administered to a patient, the composition would be affective as an immune system education and stimulatory agent to increase the overall systemic response of the host immune system. More preferably, the composition comprises an adenovirus containing a first polynucleotide encoding HSP72 and a second polynucleotide encoding GMCSF. The composition may administered in conjunction with any of the previous methods (supra) embodiments, or may be administered alone as a general immunostimulatory agent.

In yet another embodiment, a composition comprising a recombinant adenovirus expressing an immunomodulatory agent such as HSP72 or GMCSF, may be administered to a patient in conjunction with any and all vaccine, so as to make the vaccine more effective. Once expressed in the patient, HSP72 or GMCFS will effectively prime and educate the immune system to the target of the vaccine and thus increase the effectiveness of the vaccine. The composition may be administered in conjunction with a vaccine for which supply is limited, and so may be diluted and administered with neoadjuvant to allow for vaccination of a greater number of patients than would otherwise be possible.

### BRIEF DESCRIPTION OF THE DRAWINGS

FIG. 1 shows transduction efficiency of murine and rodent tumor cell lines with recombinant adenovirus.

FIG. 2 shows CTL activity from ADHSP72 treated, tumor resistant animals re-challenged with C26 cells.

FIG. 3 shows CTL activity from ADHSP72 treated, tumor resistant animals re-challenged with B16 cells.

FIG. 4 shows CTL activity from ADHSP72 treated, tumor resistant animals re-challenged with TrampC2 cells

FIG. 5 shows CTL activity from ADHSP72 treated, tumor resistant animals re-challenged with 9L cells 14 days after surgical excision of the primary tumor.

FIG. 6 shows Percent specific lysis of target cells in pooled splenocytes from 2 animals resistant to autologous tumor challenge

FIG. 7 shows Survival after neoadjuvant treatment

FIG. 8 shows Effect of neoadjuvant immuno-gene therapy on distant tumor growth and survival.

FIG. 9 shows serum levels of anti-CEA IgG antibody 7 days post vaccination.

FIG. 10 depicts serum levels of anti-CEA IgG antibody over time.

FIG. 11 depicts tumor resistance following immunization.

FIG. 12 depicts tumor volumes after challenge of immunized animals.

FIG. 13 shows ELISA SPOT assays to show effectiveness of splenocytes from each immunization against target cells TrampC2-GFP and TrampC2-CEA.

FIG. 14 shows survival of animals with pre-existing TrampC2-GFP tumors, or pre-existing TrampC2-CEA tumors following immunization.

### DETAILED DESCRIPTION OF THE INVENTION

In the simplest sense, the invention is directed to the induction of expression of an immunomodulatory molecule in a disease cell, the subsequent education of the immune system to an antigen expressed by the disease cell, and the treatment of the disease to reduce the overall disease burden in the patient, such that the chance of recurrence of expansion of the disease in the patient is significantly reduced. In this section, the inventor presents a few ways in which this basic invention may be applied. The skilled artisan will readily recognize myriad other ways to effectively practice the essence and spirit of this invention.

Although HSPs have been shown to function as an adjuvant, there has not been an efficient neoadjuvant therapy which includes transfer of the adjuvant to tumor cells, allowing for the education and priming of the host immune system, and subsequent treatment to reduce the disease burden in such a way so as enhance presentation of antigen/adjuvant complexes to host antigen presenting cells. Accordingly, a long felt need exists for a reliable and cost effective method of delivering a neoadjuvant to tumor cells prior to destruction of these cells, in such that the host will mount a heightened immune response thereby eliminating or suppressing further development of theses tumors.

### Recombinant Adenovirus as Neoadjuvant

A composition used to modulate the immune system may comprise a recombinant adenovirus containing a polynucleotide encoding an immunomodulatoy molecule, such as HSP72 or GMCSF. Immunomodulatory molecules, i.e., molecules that stimulate, prime and/or educate the immune system, are well known in the art. In a particular non-limiting example used for illustrative purposes, the adenovirus may be constructed by as follows. A cDNA for an immunomodulatory molecule can be cloned into a shuttle plasmid, which includes the left terminal sequences of an adenovirus, but is deleted of the adenovirus early region (E1 genes). After linearization, this shuttle plasmid is transfected onto 293 cells with a plasmid containing the entire adenovirus serotype 5 or 12 genome minus the left terminal region. Through homologous recombination, the recombinant adenvirus is generated.

### Intratumoral Administration of Neoadjuvant

Recombinant adenoviruses may be administered at a dose of 10⁷-10¹² pfu with relative safety if the administration is not intravenous. For neoadjuvant gene therapy, recombinant viruses can be diluted in Dextrose-5% in a total volume that is 5-30% of the total estimated volume of lesion to be injected. The recombinant virus can be administered by multiple injections by stereotaxic ultrasound guidance at approximately 1 cm increments, three-dimensionally to cover the entire lesion. Virus may be administered multiple times depending on safety and efficacy.

### Neoadjuvant Applications

The skilled artisan, who upon determining the type of cancer and an appropriate course of therapy, which includes surgical removal of the tumor, chemotherapy or other way to reduce the tumor mass, and who desires to use a neoadjuvant in conjunction with the treatment regime, would proceed as follows. The neoadjuvant preferably would be a recombinant adenovirus containing a polynucleotide encoding HSP72. The appropriate injection volume and virus titer would be determined by one skilled in the art as described above. The surgeon would then inject the composition into a primary tumor, or if more practicable, one or more secondary tumors. The surgeon will take whatever surgical efforts necessary to access the tumor within the patient. The composition is administered through direct intratumor injection, which may be accomplished stereotactically by ultrasound guidance. This surgeon would then wait a therapeutically effective time, which is expected to be preferably at least 6 hours, more preferably between 6 and 72 hours. This time allows for the adjuvant/immunomodulator (e.g., HSP72) to be expressed within the transfected tumor cells, and also for the adjuvant to form a complex with a tumor antigen. The surgeon may determine the optimal therapeutically effective time without undue experimentation, by measuring levels of HSP72 polypeptide with ELISA or similar means by tumor biopsy before or after removal of the tumor. After waiting a therapeutically effective time, the surgeon may then proceed with treatment such as surgical removal, or chemotherapy with the added benefit that the host immune system will have reached a heightened immunological state in which to defend against the disease.

In another embodiment the skilled artisan will proceed as above but included in the composition will be a polynucleotide encoding a disease specific antigen. If the disease is AIDS the antigen may be HIV. If the disease is colorectal cancer the antigen may be CEA. If the disease is melanoma, the antigen may be mark 1, or tyrosinase. The concentration of polynucleotide encoding antigen may vary with disease and severity of disease, but may be determined by one skilled in the art without undue experimentation. Levels of disease specific antigen may be determined by measuring levels of disease specific polypeptide by ELISA or similar means in the diseased tissue after tissue removal.

In another embodiment, HSP expression is stimulated through electromagnetic radiation. Radiation therapy may be used to focus radiation into a tumor. Physiological stress is known to result from radiation and may be used to trigger expression of elevated levels of HSPs, which will subsequently complex with tumor antigens within the cells. This surgeon would then wait a therapeutically effective time as describe above and proceed with treatment with the added benefit that the host immune system will be in a heightened state in which to defend against the disease.

More specifically, this invention can be directed to a method of treating breast cancer by priming the immune system against autologous tumor cells to prevent recurrence of breast cancer or to treat distant micrometastases. Potential lesions are often identified by needle or open biopsy. If cancer is detected, the patient may undergo surgical resection with or without adjuvant chemotherapy or radiotherapy. Application of neoadjuvant therapy would include the injection of a recombinant adenovirus expressing an immunomodulator, preferably a heat shock protein such as HSP72, to augment tumor immunogenicity and educate the host immune system. Administration of the neoadjuvant may be preformed by ultrasound guided needle injection directly to the tumor site. The patient would proceed with standard therapy such as surgery and, or chemotherapy or radiotherapy. The timing of neoadjuvant therapy and surgical resection will be determined without undo experimentation as reasonably determined by the surgeon or as described above. The neoadjuvant gene transfer would prime and educate the patient so that the patient may mount an anti-cancer immune response capable of affecting metastatic disease and/or preventing cancer relapse. It should be noted that the immunomodulator may be an antigen presenting molecule (e.g., MHC component, HSP), an immune cell stimulator (e.g., a cytokine such as GMCSF) or both (e.g., HSP72).

In another embodiment, the invention is directed to a composition that can be used in combination with a vaccine, to enhance the effectiveness of a week or dilute vaccine. To use this embodiment, a patient may be inoculated with a composition containing a recombinant adenovirus containing a polynucleotide encoding an immunomodulatory molecule such as HSP72 or GMCSF, and a vaccine. Following injection of the composition and expression of the immunomodulatory molecule, the immune system of the patient will then be primed for a heightened response, and educated to the specific target to which the vaccine is directed. The combination of priming and educating the immune system will thereby result in increasing the effectiveness of the vaccine. This will allow the use of reduced amounts of vaccine, or use of otherwise less effective preparations of vaccine.

By way of description but not of limitation the following terms, when used herein, following meanings. “Neoadjuvant therapy” describes therapy prior to surgery or other subsequent therapy. Neoadjuvant gene therapy utilizes direct genetic modification of a cancer cell or other disease cell prior to surgery with the aim of generating an anti-tumor (or other) immune response capable of protecting the patient against further relapse or for anti-tumor effect against metastatic disease. Neoadjuvant gene therapy can involve direct modification of the cancer cell or cancer cell microenviroment by gene transfer. Gene transfer can be accomplished by direct DNA injection, recombinant virus or other methods of introducing DNA into cells.

A “vaccine” is an agent used in immunization of a patient to result in a an immune response by the patient against the agent. Vaccines may consist of inactivated viruses, bacteria, cells or the like. Vaccines may consist of recombinant or native proteins or glycoproteins. Vaccines may be a DNA encoding a polypeptide, or a polypeptide expressed by a recombinant virus. Vaccines may be administered either intramusculary, inradermally or through direct injection into tissue including cancer tissue.

A method to augment vaccine induced immune responses entails combining the vaccine agent with an adjuvant. An adjuvant can an be an immunomodulatory molecule, which includes cytokines, chemokines, pro-inflammatory agents, and heat-shock proteins. The adjuvant can be delivered as a recombinant or purified protein, or can be encoded in a recombinant adenovirus.

Traditional therapies, to which this neoadjuvant therapy can be integrated, include vaccination, surgery, chemotherapy and the like. For cancer chemotherapy, oncolytic adenoviruses may be used, as well as more traditional forms of chemotherapy known in the art (e.g., taxol). An example oncolytic adenovirus is an adenovirus that overexpresses an adenovirus death protein.

In the practice of this invention, patients can be any vertebrate animal. The skilled artisan would reasonably expect the disclosed method and composition have varying immunological properties among vertebrates. Preferred patients in these methods are mammals; more preferred patients are humans. While any and all disease can be treated using this invention, the preferred disease is a cancer.

In addition to administration of the instant composition by intratumoral injection, the composition may be administered to a vertebrate by any other suitable route known in the art including, for example, intravenous, subcutaneous, intramuscular, transdermal, intrathecal, or intracerebral. The composition may be in a carrier, which contains pharmaceutically-acceptable excipients for modifying or maintaining the pH, osmolarity, viscosity, clarity, color, sterility, stability, rate of dissolution, rate of transfection, and odor of the formulation.

The pronouns “we” and “our”, as used in the following examples, means the inventor's research team and does not necessarily imply multiple inventors.

### EXAMPLE 1

### Neoadjuvant Immuno-GeneTherapy by Direct Intra-Tumoral Injection

### Summary

Gene modification of tumor cells is commonly utilized in various strategies of immunotherapy both as a preventative treatment and a means to modify tumor growth. Gene transfer prior to surgery as neoadjuvant therapy has not been studied systematically. We addressed if direct intra-tumoral injection of a recombinant adenovirus expressing the immunomodulatory molecule heat shock protein 72 (“ADHSP72”) administered prior to surgery could result in sustainable anti-tumor immune responses capable of affecting tumor progression and survival in a number of different murine and rat tumor models. Using intradermal murine models of melanoma (B16), colorectal carcinoma (CT26), prostate cancer (TrampC2) and a rat model of glioblastoma (9L), tumors were treated with vehicle or GFP expressing adenovirus (“ADGFP”) or ADHSP72. Tumors were surgically excised after 72 hours. Approximately 25-50% of animals in the ADHSP72 treatment group but not in control groups showed sustained resistance to subsequent tumor challenge. Tumor resistance was associated with development of anti-tumor cellular immune responses. ADHSP72 neoadjuvant therapy resulted in prolonged survival of animals upon re-challenge with autologous tumor cells compared to ADGFP or vehicle control groups. To study the effects on tumor progression of distant metastases, a single tumor focus of animals with multifocal intradermal tumors was treated. ADHSP72 diminished progression of the secondary tumor focus and prolonged survival. Our results indicate that gene modification of tumors prior to surgical intervention may be beneficial to prevent recurrence in specific circumstances.

### Methods

Adenovirus Vectors

Creation of recombinant ADHSP72 has been previously described⁴³. Briefly, the E1 region of the replication-defective adenoviral vector was replaced by an expression cassette containing the entire coding region for inducible human heat-shock protein 70 (HSP72) driven by the human CMV-IE promoter in parallel to the transcriptional direction of the adenovirus E1 ORF. ADHSP72 was generated by cloning human HSP72 from the plasmid pH2.3 (ATCC 57494, American Type Culture Collection) as a BamHI-ScaI fragment into the BamHI-PmeI sites of the adenoviral shuttle plasmid pAVC3. In a subsequent step, ADHSP72 was rescued by homologous recombination of pJM1711 and pAVC3-HSP72 in 293 cells as previously described⁴⁴. ADGFP was obtained from Dr. Maurice Green (Institute of Molecular Virology, Saint Louis University, Saint Louis, Mo.). This virus was created using the pAD-TRACK vector and the AD-EASY system according to manufacturers' protocol (Stratagene, Cedar Creek, Tex.). Both recombinant adenoviruses were propagated in 293 cells, purified by two rounds of CsCl density centrifugation, dialyzed against 1500 ml of PBS with 1 mmol/L MgCl2 and 10% glycerol four times at 4° C. (one hour each) with a Slide-A-Lyzer cassette (Pierce, Rockford, Ill.), and stored at −80° C. Virus concentration was determined by measuring absorbency at 260 nm and the titer was estimated by plaque assay on 293 cells.

Cells

A number of murine tumor cell lines were utilized in this study. B16, a murine melanoma cell line, and TrampC2, a murine prostate carcinoma cell line, are of C57B/6 mouse origin (H-2b) and were obtained from American Type Tissue Collection (ATCC, Rockville, Md.). CT26 is a commonly utilized murine colorectal tumor cell line of Balb/C origin (H-2D) also obtained from ATCC. 9L is a rat gliosarcoma chemically derived from Fisher rats and was a generous gift of Martin Graf (Medical College of Virginia, Richmond, Va.). Cells were kept at 37° C., 5% CO₂ and 95% humidity in Dulbecco's modified eagle medium (Cellgro, Herndon, Va.) supplemented with 10% (v/v) heat inactivated fetal bovine serum (BioWhittaker, Walkersville, Md.), 2 mM L-glutamine and 100 units/ml penicillin and 1000 ug/ml streptomycin (Invitrogen, Carlsbad, Calif.)

Intradermal Tumor Formation:

Cells were injected intradermally by aseptic technique on the murine or rodent dorsum after shaving in sterile phosphate buffered saline in a total volume of 50 ul. For all cell lines, 1×10⁶ cells were injected and produced on average 50-100 mm tumors in 4-7 days. In our multi-focal tumor model, an initial inoculum was given on the left dorsum. On day 5, a secondary inoculum of tumor cells were transplanted on the opposite flank.

Animal Experiments:

C57B/6 and Balb/C mice were obtained from the Jackson Laboratory (Bar Harbor, Me.) at 4-6 weeks of age. Fisher 344 rats of 150-200 grams were obtained from Charles River Laboratories (Wilmington, Mass.). All animals were acclimated to their environment for at least one week prior to experimentation. The animals were under the care of a staff veterinarian and managed in accordance with the National Institutes of Health Guide for the Care and Use of Laboratory Animals. Tumor growth was assessed by digital caliper measurement of tumors. Tumor volume was calculated as length X width². Animals were grouped by tumor size and injected with 5×10⁸ ADGFP or ADHSP72 or vehicle (phosphate buffered saline 4% sucrose v/v; PBS) once a day for two consecutive days. Tumors were surgically excised with 0.5 mm margins, 72 hours after the final injection, under aseptic technique and skin sutured using 3.0 nylon.

CTL Assay

Cytotoxicity was assessed by the ability of spleen effector cells to lyse various tumor target cells. Splenocytes were derived by mechanical disruption of spleens under aseptic conditions in PBS. Red blood cells were removed by five (5)-minute incubation in ammonium chloride lysis buffer (Pharminigen). Splenocytes were stimulated for six (6) days in 24-well tissue culture plates at a ratio of 150:1 with irradiated (8000 rads) 9L in RPMI-10% FCS supplemented with 10% Rat T-stim (Fisher Biochemicals) for rat experiments. Freshly harvested splenocytes were utilized for murine studies. Splenocytes were harvested from plates by Ficoll density centrifugation and added to 96-well U-bottom plates (Corning) in RPMI-10% FCS. For assays requiring only CD4+ or CD8+ cells, cellular fractionation was conducted using antibody column purification (Accurate Chemical). Target cells were harvested, washed in PBS and labeled with 200 uCi of ⁵¹Cr (Na₂CrO₄ in sterile saline; Amersham Biochemicals) in DMEM-10% FCS supplemented with 50 uM 2-mercaptoethanol for 90 minutes. Target cells were washed three (3) times with 10 times the volume of PBS and then added to plates containing splenocytes at the ratios described in the figure legends. After a four (4) hour incubation at 37° C., 5% CO₂, the plates were harvested using a Skatron Harvesting System (Skatron). Chromium release into supernatant was counted using an ICN gamma counter. Spontaneous Cr leakage was measured by six (6) wells that did not contain splenic effector cells. Maximum Cr leakage was determined by addition of Triton X-100 to a final concentration of 0.8% to six (6) wells. Corrected % lysis was determined by the following formula: Corrected lysis=100×(effector cell sample chromium release−spontaneous target chromium released)/(maximum target chromium released−spontaneous target chromium released).

Statistics:

Statistical analysis was performed using SPSS statistical software (Chicago, Ill.). Tumor resistance statistics was determined by Fisher exact analysis. All other data except survival was analyzed by analysis of variance. Survival analysis was performed using the Kaplan-Meier test.

### Results

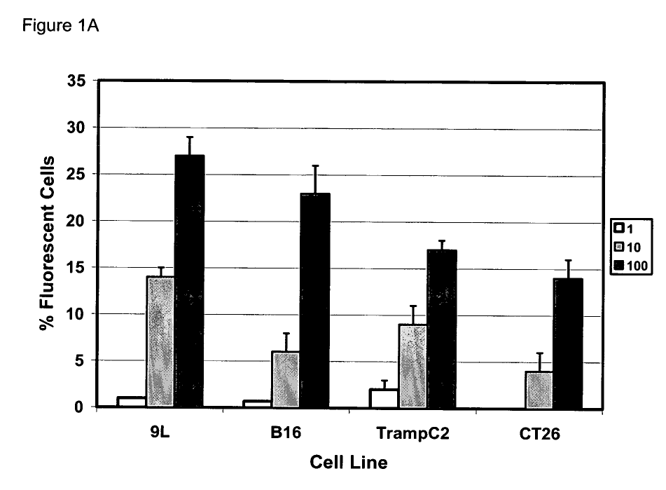

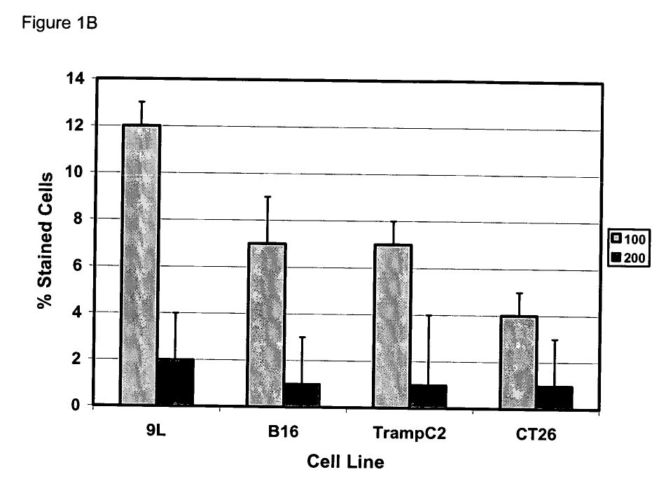

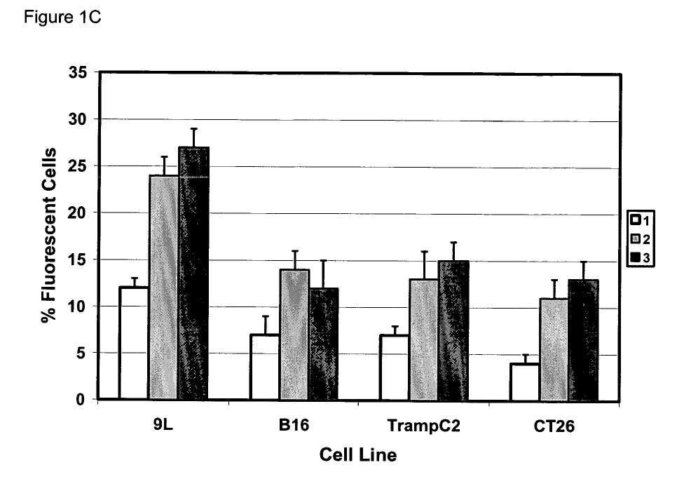

Prior to assessing the efficacy of ADHSP72 neoadjuvant immunotherapy, we determined a suitable dosage and administration method to result in effective transduction of tumor cells in situ. Direct intra-tumoral injection of recombinant adenoviruses transduced tumor cells in situ with various degrees of efficacy. In FIG. 1* we show the percentage of tumor cells transduced at various multiplicities of infection in vitro and compare it to transduction efficiency of the various tumors types in situ. Murine and rodent cells are known to infect with recombinant adenoviruses at poorer efficiency than human cells. In FIG. 1a we showed that transduction efficiency was dose dependent and that all the cell lines studied showed less than 30% transduction at a MOI of 100 in vitro. In FIG. 1b, we assessed transduction efficiency in situ. Infectivity of tumors was dependent on tumor size. At a fixed dose of 5×10⁸ pfu, smaller tumors showed more infected cells than larger tumors. The dose of 5×10⁸ was chosen based on previous experience in our laboratory with these tumor models to insure consistency ¹². Higher dosages have been utilized by others ¹³. In FIG. 1c, we assessed if multiple dosing of tumors would enhance the number of cells expressing the transgene. There was a small increase associated with injecting tumors twice. There appears not to be a benefit with further injections. Based on these experiments we opted to treat tumors of approximately 100 mm³ twice with 5×10⁸ pfu in our animal models.

*

Our main objective was to evaluate generation of anti-tumor immune responses associated with recombinant adenovirus gene transfer as neoadjuvant immunotherapy. We proceeded to inject animals with intradermal tumors with recombinant adenoviruses or vehicle control and then surgically excised the tumor after 72 hours. Animals were given a 2 week recovery period and then injected on the opposite dorsal surface with autologous tumor cells. Injection of the tumors prior to surgery did not alter tumor size in any treatment group (data not presented). Tumors progressed and were approximately 150-250 mm³ at the time of surgery. In table 1, we show the number of animals resistant to tumor formation in the various treatment groups.

Table 1 depicts the number of animals resistant to tumor formation. Animals with approximately 100 mm³ tumors were treated as described previously with the agents in the top column. Tumors were excised after 72 hours and animals were re-inoculated with autologous tumor cells 2 weeks after surgery. Positive tumor formation was determined by at minimal, palpable tumor 2 weeks from the time of tumor injection. 100% of untreated age-matched animals as a positive control were injected and showed tumor formation. Items in bold represent statistical significance by Fisher exact test (p<0.02).

TABLE 1

| | PBS | ADGFP | ADHSP72 |
|---|---|---|---|
| 9L | 0/7 | 0/8 | 8/16 |
| B16 | 0/14 | 0/15 | 6/22 |
| TrampC2 | 0/12 | 0/14 | 8/27 |
| CT26 | 0/13 | 0/12 | 8/30 |

Of note, no animals had recurrent tumor formation at the initial surgical site. Tumor resistance was only observed in the ADHSP72 treatment group. Additionally, tumor resistant animals were detected in all tumor models. Approximately 25-50% of ADHSP72 treated animals showed resistance to tumor formation in all tumor groups. This resistance to tumor formation was dependent on the initial tumor size at the time of treatment. In Table 2, we show results of an identical experiment in which the tumor size in each treatment group was on average double that of the groups shown in Table 1.

TABLE 2

| | PBS | ADGFP | ADHSP72 |
|---|---|---|---|
| 9L | 0/3 | 0/3 | 2/8 |
| B16 | 0/9 | 0/10 | 1/10 |
| TrampC2 | 0/8 | 0/9 | 1/10 |
| CT26 | 0/10 | 0/8 | 0/9 |

Table 2 depicts the number of animals resistant to tumor formation. Animals with approximately 250 mm³ tumors were treated as described previously with the agents in the top column. Tumors were excised after 72 hours and animals were re-inoculated with autologous tumor cells 2 weeks after surgery. Positive tumor formation was determined by at minimal, palpable tumor 2 weeks from the time of tumor injection. 100% of untreated age-matched animals as a positive control were injected and showed tumor formation.

Once again, tumor resistance was only detected in animals with ADHSP72 treated tumors. However, there was no significant difference between the treatment groups. The number of tumor resistant animals was less than in the previous experiment.

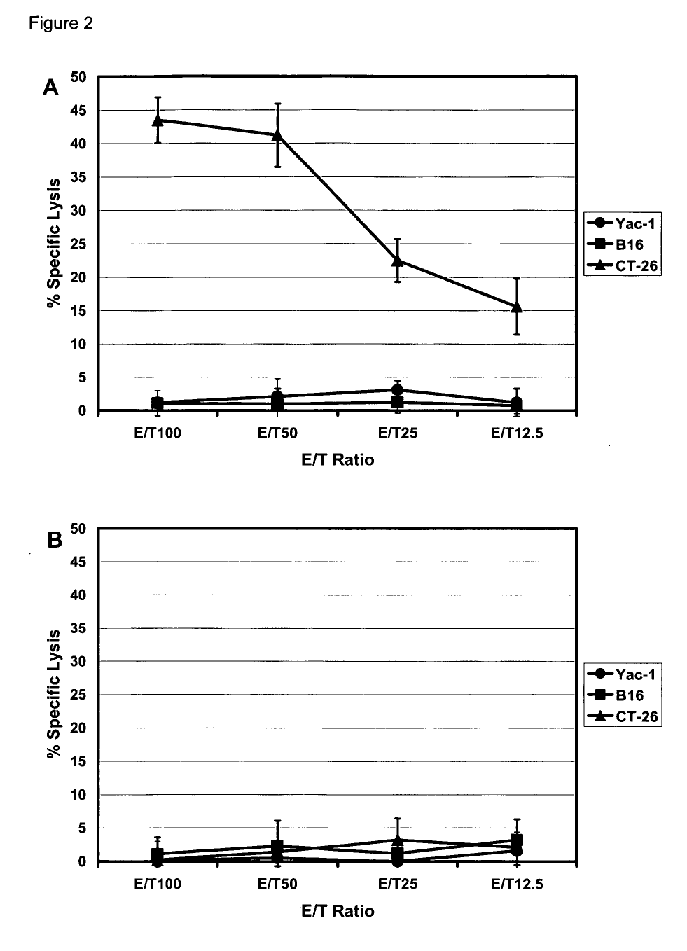

Next we determined the immunological basis of tumor resistance in these animals. In previous experiments, cellular immune responses have been detected which can confer tumor immunity to the various cell lines utilized^{13,14,15}. Splenocytes from resistant and not-resistant animals were obtained 4 weeks after the second tumor challenge. CTL activity was measured by chromium release assay. Yac-1 cells were used as a NK sensitive target cell line. Very little NK specific lysis was measured in our experiments. In FIG. 2a, we showed that CT26 tumor resistant animals had developed CT26 specific cellular immune responses. In contrast, animals without tumor resistance show lack of anti-CT26 cellular immunity (FIG. 2b).

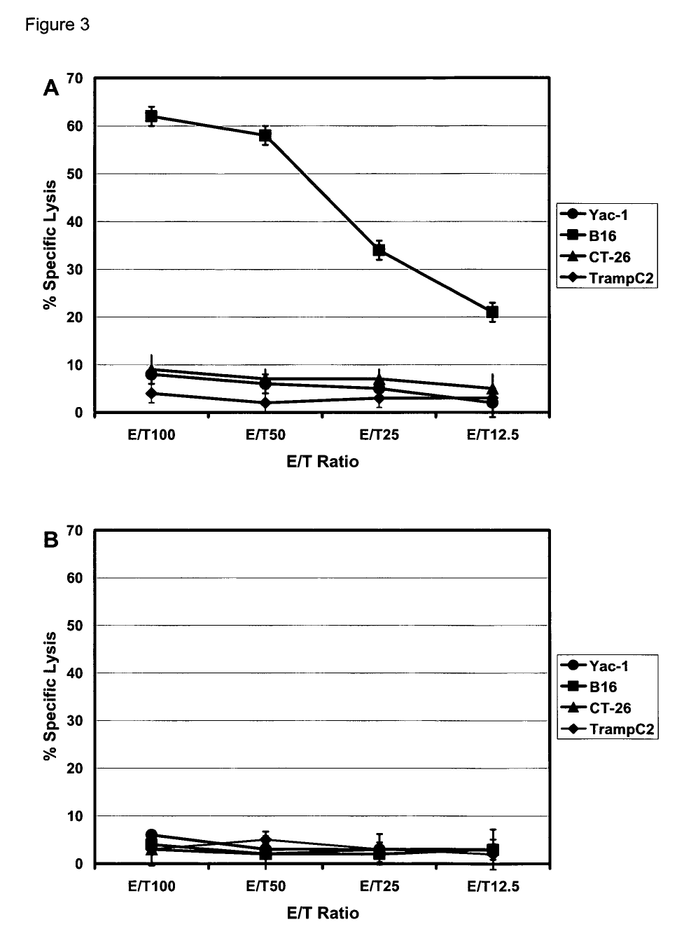

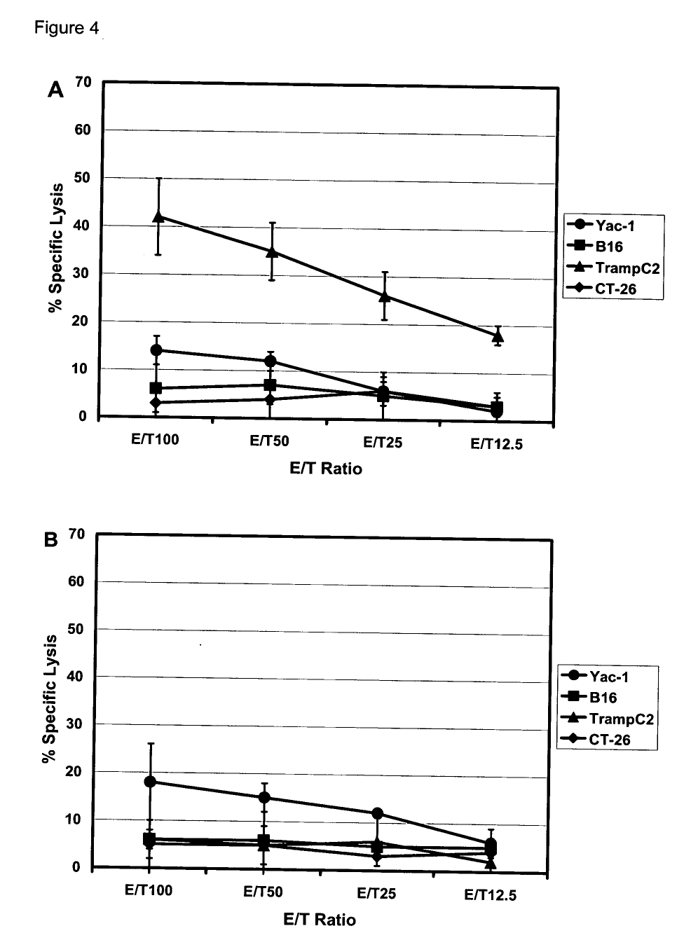

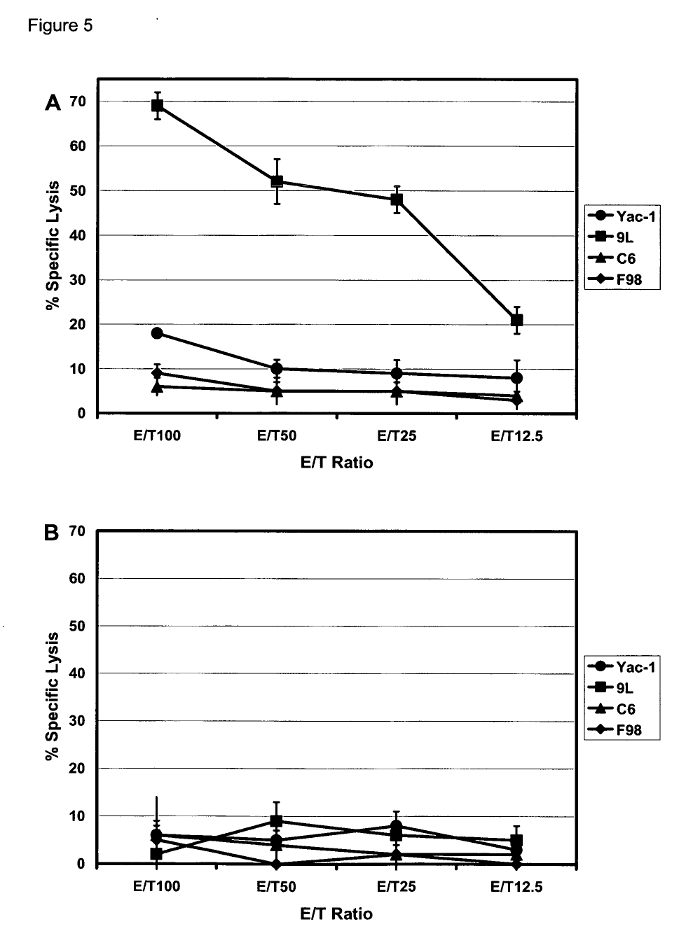

In FIG. 3a, we present data from our B16 melanoma tumor model. Similar to before, resistant animals show development of cellular immunity while non-resistant animals lack measurable cellular immune responses. Additionally, CTL responses were also tumor specific with no cross-reactivity against the syngeneic TrampC2 cell line (FIG. 3). Results were once again similar from splenocytes of ADHSP72 treated animals utilizing the TrampC2 prostate carcinoma model (FIGS. 4a and 4b). In the CT26, B16 and TrampC2 tumor models, the CTL response was MHC restricted with little lysis of the allogeneic target cell lines. These experiments in murine models show that across immunological backgrounds cellular immune responses could be generated by intra-tumoral injection of ADHSP72. Data from animals in the PBS or ADGFP treatment group in each tumor model is not represented as no measurable CTL activity was detected. We demonstrated that ADHSP72 associated tumor resistance was not limited to murine tumor models. Using an intradermal rat glioma model, we demonstrated that resistant animals had measurable tumor specific CTL activity (FIGS. 5a and 5b). The CTL activity was tumor specific and MHC restricted as in the murine models with no specific lysis of the allogeneic C6 cell line or the syngeneic F98 glioma cell line.

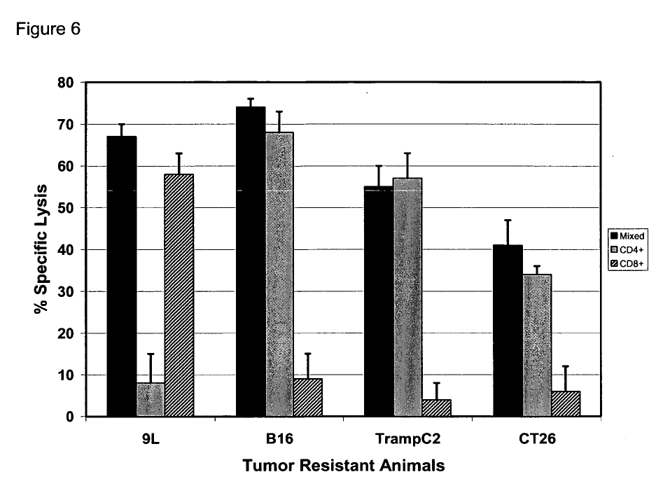

To further determine the immunological phenotype these cellular immune responses, blocking antibodies were utilized in our CTL assay. Lysis of the autologous target cell could be inhibited by anti-CD8 antibodies in the murine B16 melanoma, CT26 colorectal carcinoma, and TrampC2 prostate carcinoma models (FIG. 6). In contrast, the effector cell appears to be CD4+ in the 9L glioblastoma model (FIG. 6).

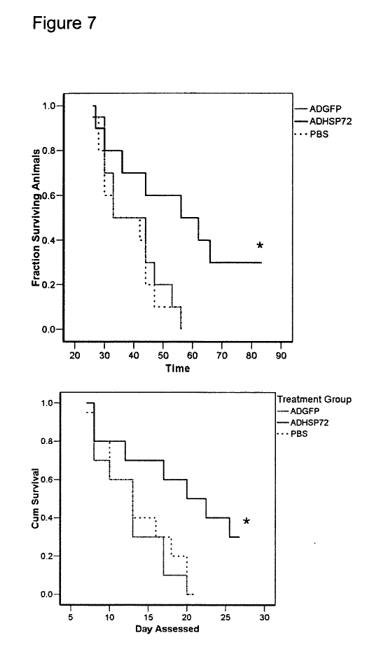

Next we addressed the effect of ADHSP72 neoadjuvant therapy on overall survival of animals. In our intradermal tumor models, the only cell line with metastatic potential was the murine prostate carcinoma cell line TrampC2¹⁶. However, in our experience, surgical excision of tumors while less than 200 mm³ did not result in metastasis or affect survival. Local or distant tumor relapse was not observed after surgical excision of primary tumors in any of the models studied. Overall survival of animals after neoadjuvant adenovirus therapy is shown in FIGS. 7a and 7b for the CT26 and B 16 tumor models respectively. Survival curves were similar for the other 2 tumor models and are not presented. In all tumor models, survival advantage is associated with ADHSP72 treatment. Vehicle or ADGFP intra-tumor injections prior to surgical excision did not confer any protection against secondary tumor challenge or prolongation of survival. The survival benefit associated with ADHSP72 treatment was related to development of anti-cellular immunity as described above.

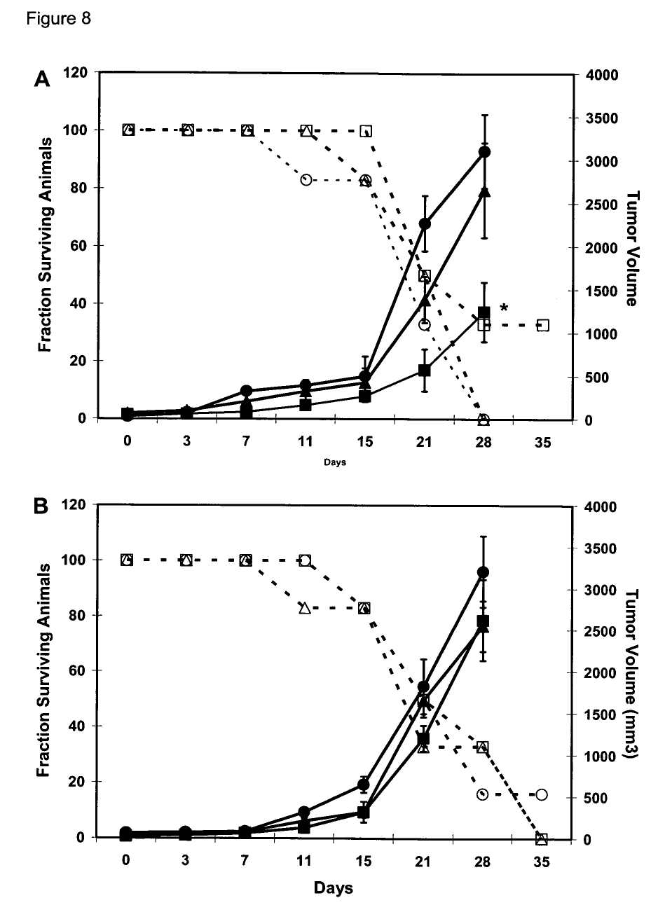

One of the difficulties with surgical therapy for many different tumors is tumor relapse. Tumor relapse is often thought to be a consequence of micrometastatic tumor not previously detected. To study the effects of neoadjuvant immunotherapy in a clinically applicable model of micrometastases we elected to treat animals with multifocal tumors. We performed these experiments in the murine models of CT26 and B16. Only a single tumor focus was treated with vehicle, ADGFP or ADHSP72. The treated tumor focus was surgically removed and tumor growth and survival of the tumor focus on the opposite flank was assessed. In FIGS. 8a and 8b we show tumor growth of B16 tumors over time and relate it to animal survival. ADHSP72 treatment of a single focus of multifocal tumors decreased tumor growth velocity of the secondary focus and prolonged survival if the secondary focus of tumor was small (<50 mm³) as shown in FIG. 8a. In contrast, (FIG. 8b), if the secondary tumor focus averaged greater than 100 mm³, there was no survival advantage and there was no difference observed in growth of distant tumor between the vehicle, ADGFP and ADHSP72 treatment groups. The data for the CT26 murine model was similar.

Figures

FIG. 1 depicts the transduction efficiency of murine and rodent tumor cell lines with recombinant adenovirus. A) ADGFP was added at various multiplicities of infection (MOI; see legend) to cell lines grown to 70% confluency in 35 mm tissue culture dishes in DMEM-2% FCS for 90 minutes followed by two (2) washes in phosphate buffered saline (PBS) and then grown overnight in complete media. After 24 hours fluorescent cells were counted on a hemacytometer using a fluorescence microscope. The percent fluorescent cells as a fraction of total cell number is given on the y-axis. Data represents results from a typical experiment. Infections were performed in triplicate. B) Intradermal tumors were injected with 5×10⁸ pfu ADGFP in total volume of 100 ul PBS. Tumors were surgically excised after 72 hours and digested in triple enzyme solution and placed in culture. Cells were evaluated for fluorescence as previously described. C) Intradermal tumors were injected numerous times with 5×10⁸ pfu ADGFP in total volume of 100 ul PBS at 24 hour intervals. Tumors were surgically excised after 72 hours and digested in triple enzyme solution and placed in culture. Cells were evaluated for fluorescence as previously described.

FIG. 2 depicts CTL activity from ADHSP72 treated, tumor resistant animals. Percent specific lysis of target cells by 4-h ⁵¹Cr-release assay is depicted on the y-axis. The splenic effector:target cell cell ratio is given on the y-axis. Effector:target incubations were performed in triplicate. Animals with intradermal CT26 tumors were treated with ADHSP72 and re-challenged with CT26 cells 14 days after surgical excision of the primary tumor. Tumor resistance was determined by the lack of palpable tumor 14 days after secondary CT26 cell inoculation. A) CTL activity from pooled splenocytes from 2 animals resistant to tumor challenge is depicted. B) CTL activity from pooled splenocytes from 2 animals not-resistant to tumor challenge is depicted.

FIG. 3 depicts CTL activity from ADHSP72 treated, tumor resistant animals. Percent specific lysis of target cells by 4-h ⁵¹Cr-release assay is depicted on the y-axis. The splenic effector:target cell cell ratio is given on the y-axis. Effector:target incubations were performed in triplicate. Animals with intradermal B16 tumors were treated with ADHSP72 and re-challenged with B16 cells 14 days after surgical excision of the primary tumor. Tumor resistance was determined by the lack of palpable tumor 14 days after secondary B16 cell inoculation. A) CTL activity from pooled splenocytes from 2 animals resistant to tumor challenge is depicted. B) CTL activity from pooled splenocytes from 2 animals not-resistant to tumor challenge is depicted.

FIG. 4 depicts CTL activity from ADHSP72 treated, tumor resistant animals. Percent specific lysis of target cells by 4-h ⁵¹Cr-release assay is depicted on the y-axis. The splenic effector:target cell cell ratio is given on the y-axis. Effector:target incubations were performed in triplicate. Animals with intradermal TrampC2 tumors were treated with ADHSP72 and re-challenged with TrampC2 cells 14 days after surgical excision of the primary tumor. Tumor resistance was determined by the lack of palpable tumor 14 days after secondary TrampC2 cell inoculation. A) CTL activity from pooled splenocytes from 2 animals resistant to tumor challenge is depicted. B) CTL activity from pooled splenocytes from 2 animals not-resistant to tumor challenge is depicted.

FIG. 5 depicts CTL activity from ADHSP72 treated, tumor resistant animals. Percent specific lysis of target cells by 4-h ⁵¹Cr-release assay is depicted on the y-axis. The splenic effector:target cell cell ratio is given on the y-axis. Effector:target incubations were performed in triplicate. Animals with intradermal 9L tumors were treated with ADHSP72 and re-challenged with 9L cells 14 days after surgical excision of the primary tumor. Tumor resistance was determined by the lack of palpable tumor 14 days after secondary 9L cell inoculation. A) CTL activity from pooled splenocytes from 2 animals resistant to tumor challenge is depicted. B) CTL activity from pooled splenocytes from 2 animals not-resistant to tumor challenge is depicted.

FIG. 6 depicts CTL Assays with Blocking Antibodies. Percent specific lysis of target cells by 4-h ⁵¹Cr-release assay is depicted on the y-axis. Pooled splenocytes from 2 animals resistant to autologous tumor challenge from the treatment groups identified on the x-axis at an effector to target ratio 100:1. Anti-CD4 or anti-CD8 antibodies (Pharmingen) were added at 30 ul/well to inhibit activity.

FIG. 7 depicts survival after neoadjuvant treatment. A) Neoadjuvant treatment of animals with intradermal CT26 tumors was performed as previously described with the vectors described in the legend. Tumors were surgically excised and animals were inoculated with CT26 cells 14 days after surgical excision and followed for survival. Data is representative of one of three independent experiments with similar results. (*, p<0.02) denotes statistical significance by Kaplan-Meier survival analysis. B) Neoadjuvant treatment of animals with intradermal B16 tumors was performed as previously described with the vectors described in the legend. Tumors were surgically excised and animals were inoculated with B16 cells 14 days after surgical excision and followed for survival. Data is representative of one of three independent experiments with similar results. (*; p<0.02) denotes statistical significance by Kaplan-Meier survival analysis.

FIG. 8 depsitcs the effect of neoadjuvant immuno-gene therapy on distant tumor growth and survival. Animals (n=10/group) were inoculated with B16 cells on opposite flanks, 5 days apart. Animals were grouped based on the size of the secondary tumor foci at the time of injection of the primary tumor. A) tumor progression (solid lines) and survival (dotted lines) of animals with secondary tumor foci<50 mm³ when the larger tumor was injected with PBS (◯,●), ADGFP (⋄,▴) or ADHSP72 (□,▪) and surgically excised after 72 hours. B) tumor progression (solid lines) and survival (dotted lines) of animals with secondary tumor foci>50 mm³ when the larger tumor was injected with PBS (◯,●), ADGFP (⋄,▴) or ADHSP72 (□,▪) and surgically excised after 72 hours. (*) denotes statistical significance by ANOVA.

### Discussion

Surgical resection is a common treatment modality for many different types of tumors. Unfortunately, diagnostic modalities do not allow for the detection of micrometastatic disease and many patients relapse with disease after surgical excision of the primary focus.

Consequently, methods of treating micrometastatic disease may be efficacious in preventing tumor relapse when the primary treatment method is surgical resection. Neoadjuvant immunotherapy is aimed at generating anti-tumor immune responses for that purpose. To date, neoadjuvant immunotherapy has been limited to the use of recombinant biologicals ⁴. However, in animal models, gene transfer has been shown to be a potent method of developing anti-tumor immunity. A number of different agents have been utilized and aim at either increasing tumor immunogenicity or modifying immune effector cell recognition of tumor cells. Agents aimed at increasing tumor immunogenicity include ICAM-1 ¹⁷, B7-1 ¹⁸ and B7-2 ¹⁹ and CD40 ligand ²⁰. The second category of immune system modifying genes transferred include cytokines such as GM-CSF ²¹, IL-2 ²², IL-4 ²³, IL-12 ²⁴ and RANTES ³.

In experimental immunotherapy studies the vehicle for gene transfer has varied from chemical methods using lipid-gene complexes, electroporation and virus based methods. In this study we utilized a recombinant adenovirus. Adenovirus vectors remain episomal in infected cells reducing the risk of genomic integration and subsequent oncogenesis. Additionally, adenoviruses are immunogenic ²⁵ and may augment immune response generation, a property ideal for cancer gene therapy. Gene expression is transient and if the transgene holds no toxic properties, the transferred gene should have limited long term effects.

In this study, we used a recombinant adenovirus to transfer the HSP72 gene to cancer cells in various experimental murine and rodent tumor models. The HSP72 gene has previously been shown to increase tumor immunogenicity by acting as a co-stimulatory molecule ²⁶. Tumors engineered to overexpress HSP72 have altered tumorigenic potential compared to wild-type tumors ²⁷. HSP72 is also a molecular chaperone capable of binding tumor antigens and presenting to antigen presenting cells to result in anti-tumor cellular immune responses ²⁸. In animal models, purified HSP72-antigen complexes for vaccination have resulted in sustained anti-tumor immune responses against autologous tumor cells ²⁹. Additionally, fusion proteins of HSP72 ¹² and antigens or recombinant HSP72 ³⁰ and purified antigens have been utilized in immunotherapy studies with success. The limitations to this method are the time intensive nature of HSP72 purification and the requirement for known antigens. Additionally, knowledge of the immunodominant antigenic peptides restricted to specific MHC class must be present. Subsequently, we have utilized direct HSP72 gene transfer into tumors where it can bind specific antigenic peptides. Another advantage to direct HSP72 gene transfer is the immunostimulatory properties of this molecule. HSP72 stimulates cytokine secretion from monocytes ³¹ and induces the maturation of dendritic cells through the CD91 receptor ³² serving a paracrine function in attraction of immune effector cells. Additionally, HSP72 expression in poorly immunogenic cell lines like the murine B16 melanoma cell line significantly up-regulated MHC class I expression ³³. A similar effect of HSP72 has been reported in the 9L glioblastoma cell line ³⁴. Therefore, direct intra-tumor injection can directly increase tumor immunogenicity. In our study, we included the B16 and 9L cell lines as well as the CT26 murine colorectal cancer cell line. All of these cell lines are poorly immunogenic and naïve tumor cell vaccination is incapable of generating anti-tumor immunity.

The most important finding in our study was that direct intra-tumor HSP72 gene transfer prior to surgical resection had the capability of generating anti-tumor immune responses protective against subsequent tumor challenge. Previously it has been established that direct intra-tumor HSP72 gene transfer was effective in generation of anti-tumor immune responses through increased intra-tumoral infiltration of T cells, macrophages and dendritic cells and induction of a Th1 cytokine profile ³¹. In our study of neoadjuvant gene transfer immunotherapy using various experimental animal tumor models with varying immunogenicity, direct intra-tumoral ADHSP72 injection prior to surgical resection of tumors resulted in generation of cellular immune responses in approximately 25-50% of animals depending on the tumor model studied. Immune response generation was dependent on HSP72 gene transfer as adenovirus mediated GFP gene transfer had no ability to confer protection against secondary tumor challenge. We may also conclude that the immunogenicity of the backbone adenovirus vector was not sufficient to prime immune response generation. Cellular immune responses were CD8+ T-cell mediated in the murine models and CD4+ T-cell mediated in the rat glioblastoma model.

We identified one factor associated with immune response generation which was initial tumor size. Treatment of larger initial tumors was less efficacious as neoadjuvant therapy.

The purpose of this study was to evaluate the utility of HSP72 gene transfer for neoadjuvant immunotherapy. We showed ADHSP72 to be effective in prevention of tumor formation upon rechallenge of animals with tumor cells 14 days following surgical excision. ADHSP72 was also effective in altering growth of distant tumor and modifying survival dependent on the size of the distant tumor. Growth of small distant tumor foci could be altered. A number of factors may be responsible and require further study. Clearly there was an association between tumor burden and probability of anti-tumor response generation. We hypothesize that generalized immunosuppresion related to tumor burden may be a dependent factor on immune response generation. In the rat 9L model, we have previously evaluated this hypothesis by studying proliferation of splenocytes in response to mitogens from animals after surgical resection. Compared to non-tumor bearing animals, there was diminished T-cell responses to mitogens for approximately 3 weeks following surgical excision ³⁵. In a model system with multiple tumor foci, animals with surgical resection of a single tumor focus continued to have diminished T-cell responses with no normalization. Another factor associated with development of anti-tumor immunity is the induction of tumor cell apoptosis or necrosis ³⁶. HSP72 as a molecular chaperone binds antigenic peptides which when liberated from the tumor cell intracellular compartment can activate antigen presenting cells.

Another issue which may confound the efficacy of ADHSP72 treatment in our model is the timing of treatment. Surgical resection was conducted 72 hours after intra-tumoral injection of recombinant vectors. Although we did not study timing of gene expression in situ, we have fair evidence from our in vitro studies that maximal gene expression was seen by 72 hours. However, for generation of effective cellular immune responses the requirement is for gene transfer, antigen presentation to antigen presenting cells, priming of cytotoxic T-cells and migration of memory CTL outside of the tumor. In various other gene transfer strategies employing direct intra-tumoral recombinant virus injection, active TILs with cytotoxic capacity against tumor cells can be generated ³⁸. The factors governing migration of memory effector cells, whether APCs or activated T-lymphocytes have not been determined.

Cancer gene therapy has been considered a novel method for the treatment of various cancers. Genes transferred have included those directly affecting the growth of tumor cells such as p53 ³⁹ or RB ⁴⁰, genes which increase the susceptibility of tumor cells to a chemotherapeutic agent such as ganciclovir ⁴¹ or sensitize cells to radiotherapy ⁴² or genes which augment generation of anti-tumor immune responses ²³. Disadvantages of the non-immunotherapeutic methods is the need to modify 100% of cells to have the desired effect. In contrast, immunotherapy requires activation of the immune response through either modifying tumor antigen expression and/or presentation or activation of immune effector cells. We have studied gene transfer in the novel setting of neoadjuvant immunotherapy. We elected to use the HSP72 gene for its abilities to augment antigen presentation as well as its immunostimulatory properties. HSP72 gene transfer is not dependent on knowledge of the tumor-specific antigen and should be capable of generating tumor-specific immune responses against most immunogenic solid tumors. Adenovirus mediated HSP72 gene transfer for neoadjuvant immunotherapy was effective in protecting animals from secondary tumor challenges and increasing survival in various different experimental tumor models suggesting it may be an effective strategy for the treatment of tumors where there is a concern for relapse due to micrometastatic disease. Our study suggests that neoadjuvant gene transfer immunotherapy may be useful as an alternative application of cancer gene therapy.

### Literature Cited in the Example

The following references are cited by number in this example. Applicant reserves the right to challenge the veracity of statements made in these references.
1 Quan W D, Jr., Palackdharry C S. Common cancers—immunotherapy and multidisciplinary therapy: Parts III and IV. Dis Mon 1997; 43: 745-808.
2 Morse M A et al. The feasibility and safety of immunotherapy with dendritic cells loaded with CEA mRNA following neoadjuvant chemoradiotherapy and resection of pancreatic cancer. Int J Gastrointest Cancer 2002; 32: 1-6.
3 Delman K A et al. Efficacy of multiagent herpes simplex virus amplicon-mediated immunotherapy as adjuvant treatment for experimental hepatic cancer. Ann Surg 2002; 236: 337-342; discussion 342-333.
4 Buter J, Pinedo H M. Neoadjuvant chemoimmunotherapy in locally advanced breast cancer: a new avenue to be explored. Curr Oncol Rep 2003; 5: 171-176.
5 Bodar E et al. Immunologic and biologic properties of the primary tumor during prolonged neoadjuvant chemoimmunotherapy. Oncology (Huntingt) 2002; 16: 32-39.
6 Sabel M S et al. Intratumoral IL-12 and TNF-alpha-loaded microspheres lead to regression of breast cancer and systemic antitumor immunity. Ann Surg Oncol 2004; 11: 147-156.
7 Udono H, Srivastava P K. Heat shock protein 70-associated peptides elicit specific cancer immunity. J Exp Med 1993; 178: 1391-1396.
8 Blachere N E, Srivastava P K. Heat shock protein-based cancer vaccines and related thoughts on immunogenicity of human tumors. Semin Cancer Biol 1995; 6: 349-355.
9 Li Z, Menoret A, Srivastava P. Roles of heat-shock proteins in antigen presentation and cross-presentation. Curr Opin Immunol 2002; 14: 45-51.
10 Benihoud K, Yeh P, Perricaudet M. Adenovirus vectors for gene delivery. Curr Opin Biotechnol 1999; 10: 440-447.
11 Chuah M K, Collen D, VandenDriessche T. Biosafety of adenoviral vectors. Curr Gene Ther 2003; 3: 527-543.
12 Huang C et al. Potent antitumor effect elicited by superantigen-linked tumor cells transduced with heat shock protein 70 gene. Cancer Sci 2004; 95: 160-167.
13 Okada T et al. AV.TK-mediated killing of subcutaneous tumors in situ results in effective immunization against established secondary intracranial tumor deposits. Gene Ther 2001; 8: 1315-1322.
14 Xu D et al. NK and CD8+ T cell-mediated eradication of poorly immunogenic B16-F10 melanoma by the combined action of IL-12 gene therapy and 4-1BB costimulation. Int J Cancer 2004; 109: 499-506.
15 Kikuchi T, Moore M A, Crystal R G. Dendritic cells modified to express CD40 ligand elicit therapeutic immunity against preexisting murine tumors. Blood 2000; 96: 91-99.
16 Kwon E D et al. Elimination of residual metastatic prostate cancer after surgery and adjunctive cytotoxic T lymphocyte-associated antigen 4 (CTLA-4) blockade immunotherapy. Proc Natl Acad Sci USA 1999; 96: 15074-15079.
17 D'Angelica M et al. Herpes simplex virus (HSV)-mediated ICAM-1 gene transfer abrogates tumorigenicity and induces anti-tumor immunity. Mol Med 1999; 5: 606-616.
18 Townsend S E, Su F W, Atherton J M, Allison J P. Specificity and longevity of antitumor immune responses induced by B7-transfected tumors. Cancer Res 1994; 54: 6477-6483.
19 Martin-Fontecha A et al. Heterogeneous effects of B7-1 and B7-2 in the induction of both protective and therapeutic anti-tumor immunity against different mouse tumors. Eur J Immunol 1996; 26: 1851-1859.
20 Kikuchi T, Crystal R G. Anti-tumor immunity induced by in vivo adenovirus vector-mediated expression of CD40 ligand in tumor cells. Hum Gene Ther 1999; 10: 1375-1387.
21 Lee C T et al. Genetic immunotherapy of established tumors with adenovirus-murine granulocyte-macrophage colony-stimulating factor. Hum Gene Ther 1997; 8: 187-193.
22 Brockstedt D G et al. Development of anti-tumor immunity against a non-immunogenic mammary carcinoma through in vivo somatic GM-CSF, IL-2, and HSVtk combination gene therapy. Mol Ther 2002; 6: 627-636.
23 Park K H et al. Gene therapy with GM-CSF, interleukin-4 and herpes simplex virus thymidine kinase shows strong antitumor effect on lung cancer. Anticancer Res 2003; 23: 1559-1564.
24 Mazzolini G, Prieto J, Melero I. Gene therapy of cancer with interleukin-12. Curr Pharm Des 2003; 9: 1981-1991.
25 Schagen F H, Ossevoort M, Toes R E, Hoeben R C. Immune responses against adenoviral vectors and their transgene products: a review of strategies for evasion. Crit Rev Oncol Hematol 2004; 50: 51-70.
26 Clark P R, Menoret A. The inducible Hsp70 as a marker of tumor immunogenicity. Cell Stress Chaperones 2001; 6: 121-125.
27 Massa C et al. Enhanced efficacy of tumor cell vaccines transfected with secretable hsp70. Cancer Res 2004; 64: 1502-1508.
28 Blachere N E et al. Heat shock protein vaccines against cancer. J Immunother 1993; 14: 352-356.
29 Todryk S M, Gough M J, Pockley A G. Facets of heat shock protein 70 show immunotherapeutic potential. Immunology 2003; 110: 1-9.
30 Ito A, Matsuoka F, Honda H, Kobayashi T. Antitumor effects of combined therapy of recombinant heat shock protein 70 and hyperthermia using magnetic nanoparticles in an experimental subcutaneous murine melanoma. Cancer Immunol Immunother 2004; 53: 26-32.
31 Todryk S et al. Heat shock protein 70 induced during tumor cell killing induces Th1 cytokines and targets immature dendritic cell precursors to enhance antigen uptake. J Immunol 1999; 163: 1398-1408.
32 Basu S, Binder R J, Ramalingam T, Srivastava P K. CD91 is a common receptor for heat shock proteins gp96, hsp90, hsp70, and calreticulin. Immunity 2001; 14: 303-313.
33 Wells A D et al. Hsp72-mediated augmentation of MHC class I surface expression and endogenous antigen presentation. Int Immunol 1998; 10: 609-617.
34 Ito A et al. Augmentation of MHC class I antigen presentation via heat shock protein expression by hyperthermia. Cancer Immunol Immunother 2001; 50: 515-522.
35 Shah M. Tumor Imunity Following Adneovirus Mediated Herpes Simplex Thymidine Kinase Gene Transfer to Experimental Rat Gliomas. Anatomy and Neurobiology. Medical College of Virginia: Richmond, 1996.
36 den Brok M H et al. In situ tumor ablation creates an antigen source for the generation of antitumor immunity. Cancer Res 2004; 64: 4024-4029.
37 Huang X F et al. A broadly applicable, personalized heat shock protein-mediated oncolytic tumor vaccine. Cancer Res 2003; 63: 7321-7329.
38 Mulders P et al. Adenovirus-mediated interleukin-2 production by tumors induces growth of cytotoxic tumor-infiltrating lymphocytes against human renal cell carcinoma. J Immunother 1998; 21: 170-180.
39 Wolf J K et al. A phase I study of Adp53 (INGN 201; ADVEXIN) for patients with platinum- and paclitaxel-resistant epithelial ovarian cancer. Gynecol Oncol 2004; 94: 442-448.
40 Ip S M, Huang T G, Yeung W S, Ngan H Y. pRb-expressing adenovirus Ad5-Rb attenuates the p53-induced apoptosis in cervical cancer cell lines. Eur J Cancer 2001; 37: 2475-2483.
41 Vassaux G, Martin-Duque P. Use of suicide genes for cancer gene therapy: study of the different approaches. Expert Opin Biol Ther 2004; 4: 519-530.
42 Rosenberg E et al. Radiosensitization of human glioma cells in vitro and in vivo with acyclovir and mutant HSV-TK75 expressed from adenovirus. Int J Radiat Oncol Biol Phys 2002; 52: 831-836.
43 Okubo S et al. Gene transfer of heat-shock protein 70 reduces infarct size in vivo after ischemia/reperfusion in the rabbit heart. Circulation 2001; 103: 877-881.
44 Graham F L, Smiley J, Russell W C, Nairn R. Characteristics of a human cell line transformed by DNA from human adenovirus type 5. J Gen Virol 1977; 36: 59-74.

### EXAMPLE 2

### Combined Adenovirus-HSP72 and CEA-Plasmid Vaccination

### Summary

This particular example studies the effects of recombinant adenoviruses as immune adjuvants for DNA vaccination. In a mouse model, using the weak immunogen carcinoembryonic antigen (CEA), anti-CEA IgG production was significantly higher and occurred earlier when immunization included a recombinant adenovirus together with CEA-plasmid DNA. Combined immunization with a recombinant adenovirus expressing the immunomodulatory molecule heat shock protein 72 (ADHSP72) and CEA-plasmid DNA resulted in CEA-specific T-cell activation capable of protecting mice from tumor formation with CEA expressing cells. Additionally, animals with CEA expressing tumors showed diminished tumor growth and prolonged survival when immunized with ADHSP72 and CEA-plasmid DNA compared to controls. Recombinant adenoviruses expressing immunomodulatory molecules such as HSP72 may be useful adjuvants for DNA vaccination.

### Methods

Construction of Plasmids

The plasmid pCMV-CEA was created as previously described [3]. As immunological responses have previously been reported against the neomycin gene, this plasmid lacks any antibiotic resistance sequences. In contrast, in order to create a stably transfected cell line, CEA cDNA sequence was subsequently digested with Nhe 1 and Xba 1 and ligated to reciprocally digested plasmid pEGFP-N2 (Clontech, Palo Alto, Calif.) lacking the GFP sequence but maintaining the neomycin resistance gene and called pEGFP-CEA.

Generation of Replication-Defective Recombinant Adenovirus

Creation of recombinant ADHSP72 has been previously described [14]. Briefly, the E1 region of the replication-defective adenoviral vector was replaced by an expression cassette containing the entire coding region for inducible human heat-shock protein 70 (HSP72) driven by the human CMV-IE promoter in parallel to the transcriptional direction of the adenovirus E1 ORF. ADHSP72 was generated by cloning human HSP72 from the plasmid pH2.3 (ATCC 57494, American Type Culture Collection) as a BamHI-ScaI fragment into the BamHIPmeI sites of the adenoviral shuttle plasmid pAVC3. In a subsequent step, ADHSP72 was rescued by homologous recombination of pJM1711 and pAVC3-HSP72 in 293 cells as previously described [14]. ADGFP was obtained from Dr. Maurice Green (Institute of Molecular Virology, Saint Louis University, Saint Louis, Mo.). This virus was created using the pAD-TRACK vector and the AD-EASY system according to manufacturers' protocol (Stratagene, Cedar Creek, Tex.). Both recombinant adenoviruses were propagated in 293 cells, purified by 2 rounds of CsCl density centrifugation [15], dialyzed against 1500 ml of PBS with 1 mmol/L MgCl2 and 10% glycerol 4 times at 4° C. (1 hour each) with a Slide-A-Lyzer cassette (Pierce, Rockford, Ill.), and stored at −80° C. Virus concentration was determined by measuring absorbency at 260 nm [16] and the titer was estimated by plaque assay on 293 cells.

Animals

Female C57B/6 mice at 8 weeks of age were purchased from Charles River Laboratories (Wilmington, Mass.). Animals were housed at the Saint Louis University comparative medicine facility, 5 per cage, in filter topped cages with standard rodent diet under biosafety level 2 precautions. Animals were allowed to acclimate for 2 weeks prior to experimentation. The animals were under the care of a staff veterinarian and managed in accordance with the National Institutes of Health Guide for the Care and Use of Laboratory Animals.

Immunization

Animals were vaccinated intramuscularly three times at weekly intervals. Vaccinations occurred in the femoral muscle with 50 ug of plasmid DNA or 50 ug of plasmid DNA and 10⁸ recombinant adenovirus in a total volume of 50 ul established with ice-cold phosphate buffered saline.

Cell Lines

Tramp-C2 was obtained from ATCC(CRL-2731). 293 cells were obtained from Dr. Maurice Green (Institute of Molecular Virology, Saint Louis University). Cells were kept at 37° C., 5% CO₂ and 95% humidity in Dulbecco's modified eagle medium (Cellgro, Herndon, Va.) supplemented with 10% (v/v) heat inactivated fetal bovine serum (BioWhittaker, Walkersville, Md.), 2 mM L-glutamine and 100 units/ml penicillin and 1000 ug/ml streptomycin (Invitrogen, Carlsbad, Calif.). TrampC2-CEA cells were created by transfection of TrampC2 cells with the plasmid pEGFP-CEA using lipofectamine 2000 (Invitrogen, Carlsbad, Calif.) according to the manufacturer's protocol. CEA expression was ascertained by standard western blot analysis (data not shown) using anti-CEA (Biomeda, Foster City, Calif.) and peroxidase labeled goat anti-mouse secondary antibody from cell extracts run on a 10% SDS-PAGE gel and transferred to PVDF membrane. TrampC2-GFP cells were created similarly by transfection with the pEGFP-N2 plasmid to control for effects from other plasmid elements such as the antibiotic resistance gene neomycin.

Quantitation of CEA Antibody

Anti-CEA antibodies were detected using a cellular enzyme linked immunosorbent assay (ELISA) as previously described [4]. Briefly, 96-well plates were coated with 2×10⁵ 293 cells or 293-CEA cells by fixation using 0.5% Formalin and incubated at 4° C. overnight. Wells were washed with phosphate buffered saline (PBS) containing 0.5% Tween 20 and blocked for 2 hours with PBS-2% bovine serum albumin. Diluted serum samples or geometric dilutions of anti-CEA antibody as a positive control were added to plates for 2 hours at room temperature. After washing with PBS, horse-radish peroxidase labeled goat anti-mouse IgG in PBS-1% was added for 1 hour. Wells were rinsed with PBS five times followed by a single wash in carbonate buffer (pH 9.6). Reaction was developed using 4% 0-phenylenediamine dihydrochloride and read at A490 by spectrophotometry. Concentrations of anti-CEA antibody were calculated based on the standard curve developed from the positive control dilution wells. Results are reported as units where 1 U of anti-CEA antibody is equivalent to 1 ug of anti-CEA monoclonal antibody.

ELISA SPOT Assay

Multi-screen-HA plates (Millipore, Bedford, Mass.) were coated overnight at 4° C. with 10 ug/ml anti-murine IFN-g mAb (Mabtech, Stockholm, Sweden) in carbonate-bicarbonate buffer (pH9.6). Plates were blocked with RPMI 1640 and 10% fetal calf serum for 2 hours at 37° C. Splenocytes from treated animals were obtained by mechanical disruption and purified by density centrifugation. Wells were seeded with 10⁵ splenocytes and Mitomycin C treated target cells in triplicate. Plates were incubated overnight at 37° C., 5% CO₂ and 95% humidity and cells were removed by washing with PBS-0.05% Tween20 (Sigma, St. Louis, Mo.). Biotinylated anti-murine IFN antibody at a concentration of 1 ug/ml in PBS-0.5% fetal calf serum was added and plates incubated for 2 hours at 37° C. Streptavidin-conjugated alkaline phosphatase was added for one hour and spot development induced for 10 minutes with 5-bromo-4-chloro-3indolyphsphate and nitroblue tetrazolium. Spots were counted manually under an inverted microscope.

Statistics

Statistical analysis was performed using SPSS statistical software (Chicago, Ill.). All data except survival was analyzed by analysis of variance. Survival analysis was performed using the Kaplan-Meier test.

### Results

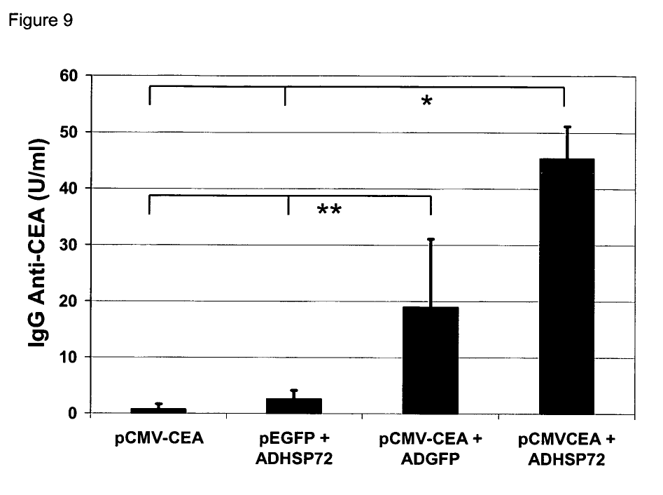

Previously it was reported that direct intramuscular injection of CEA containing plasmids at 100 ug/animal required at least four (4) vaccine administrations at weekly intervals prior to detecting measurable CTL responses [17]. Two DNA injections were required to detect anti-CEA antibodies but consistent antibody titers were not detected until after 4 or 5 injections [4]. To study the adjuvant effects of concomitant adenovirus administration, we collected serum from animals on a weekly basis, one week following vaccination. Measurement of anti-CEA IgG one week following vaccination is shown in FIG. 9. The CEA-plasmid vaccination group or the recombinant adenovirus plus control plasmid group did not result in significant anti-CEA IgG antibody titers. In contrast, CEA-plasmid vaccination with recombinant adenovirus resulted in significant titers of anti-CEA antibodies. An equivalent amount of anti-CEA IgG was measured in groups receiving CEA-plasmid and ADGFP or ADHSP72. Antibody titers in these groups were significantly elevated compared to CEA-plasmid vaccination alone.

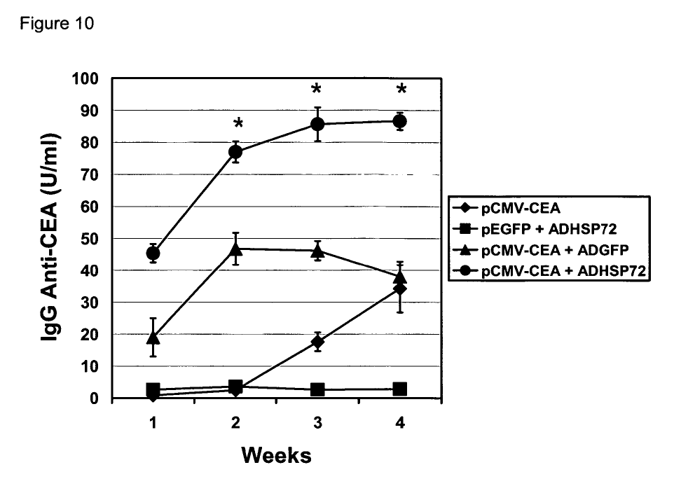

In FIG. 10 we analyzed anti-CEA IgG production over time with repeated vaccine administration. Anti-CEA IgG levels did not rise until week 3 (after 3 vaccinations) and only after 4 vaccinations was there a significant antibody titer when compared to earlier time points. A maximal antibody titer of 34 U/ml is similar to a maximal titer (38 U/ml) measured with ADGFP and CEA-plasmid together. However, the use of recombinant ADGFP with CEA-plasmid resulted in this level of antibody titer after the second vaccine administration. Antibody titers in this group remained constant after the second dose and did not change significantly with subsequent doses. Primarily, our hypothesis was concerned with the adjuvant effects of HSP72 to augment DNA vaccination. Similar to the vaccination strategy with ADGFP and CEA-plasmid administration, anti-CEA IgG antibodies were detected after a single treatment and rose dramatically after a second administration. Subsequent vaccinations did not result in a significant increase in antibody titers although the trend appears to increase marginally with each subsequent administration with a plateau after the third treatment. In FIG. 2, a noticeably higher titer of anti-CEA IgG was generated at all time points with ADHSP72 and CEA-plasmid immunization compared to ADGFP and CEA-plasmid immunization or CEA-plasmid alone. ADHSP72 in conjunction with CEA-plasmid immunization resulted in significantly higher titers than all other groups after the second immunization. HSP72 expression appears to augment the general adjuvant effect of adenovirus administration and promote antigen specific IgG production.

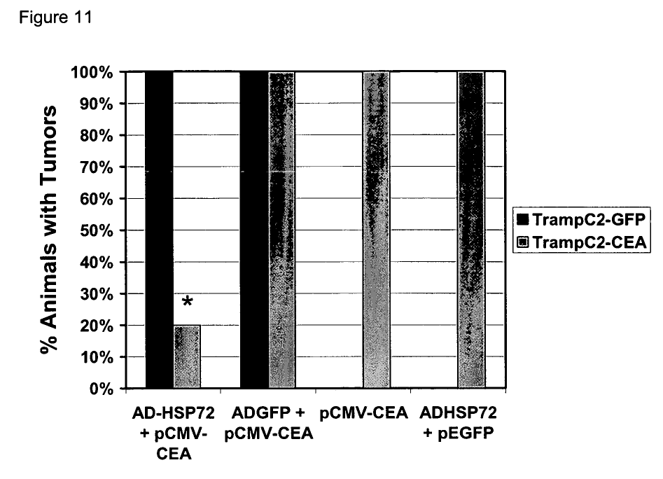

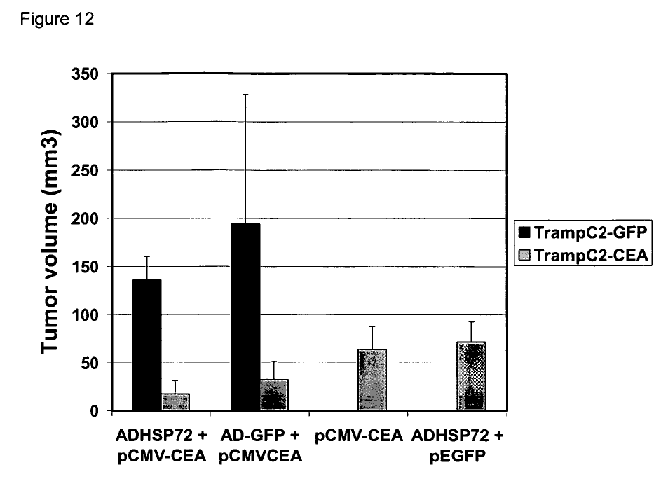

We next addressed the efficacy of the various vaccination strategies in terms of anti-tumor effect. We elected to immunize animals at weekly intervals for three weeks. Subsequently, we challenged animals with subcutaneous tumor inoculations with either TrampC2-GFP or TrampC2-CEA cells. All animals challenged with TrampC2-GFP cells showed tumor formation as shown in FIG. 11. No immunization strategy conferred resistance to TrampC2-GFP tumor cell challenge. In contrast, TrampC2-CEA formed tumors in the ADHSP72+pEGFP, pCMV-CEA alone, and ADGFP+pCMV-CEA groups. However, eighty percent of the animals immunized with ADHSP72 and CEA-plasmid showed resistance to tumor formation. During the course of this experiment we measured tumor volumes after one month (FIG. 12). TrampC2 is a syngeneic prostate cancer cell line with metastatic potential. Consequently, subcutaneous growth shows significant variability in our experience. Regardless of immunization strategy, TrampC2-GFP cells grew more rapidly than TrampC2-CEA cells as evidenced by the greater tumor volume (FIG. 12). In vitro they showed similar growth characteristics and division times (data not presented). While not wishing to be bound by theory, we hypothesize that CEA as a xenogeneic tumor antigen has some interaction with host immunity to affect growth characteristics. No homologous murine genes exist and our study was designed to study immune responses against a xenoantigen. Importantly, in the control groups 100% of animals formed tumors with TrampC2-CEA inoculation. There was little effect of the neomycin antibiotic resistance gene as both TrampC2-GFP and TrampC2-CEA contain this gene in the backbone plasmids.

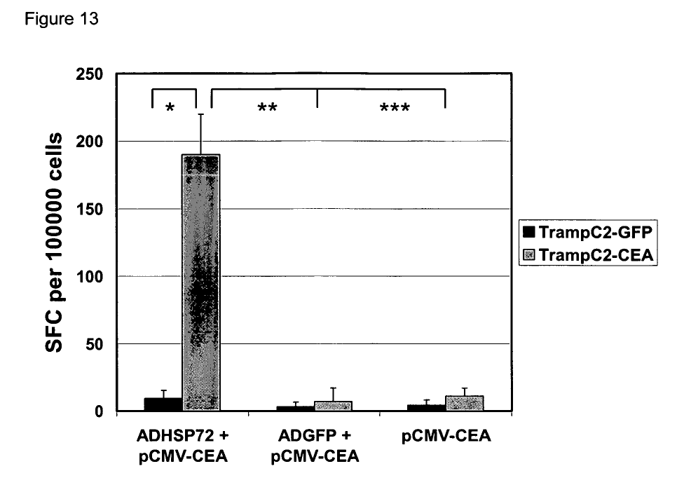

To assess the specificity of the immune response and to characterize the generation of cytotoxic T-lymphocytes, animals resistant to TrampC2-CEA inoculation were rechallenged with TrampC2-CEA or TrampC2-GFP tumor cells one month after the initial tumor challenge. All animals were resistant to this subsequent challenge with TrampC2-CEA but not to TrampC2-GFP tumor cells indicating the presence of a long-term memory immune response with specificity for tumor cells expressing CEA. To further study cellular immune response generation, assays were conducted from animals following the final immunization. In FIG. 13, we show that T-cell activation to TrampC2-CEA cells is present only in the ADHSP72+CEA-plasmid immunization group. There is a statistically significant number of activated spot forming cells in the ADHSP72+CEA-plasmid group compared to all other groups. This T-cell response is specific for CEA as very little activation is measured upon incubation of effector cells with the TrampC2-GFP target cells. Once again, the neomycin antibiotic resistance gene present in both cell lines is not recognized by effector cells. Interestingly, very little T-cell activation is measured from the ADGFP+CEA-plasmid immunization group. The adjuvant effects of recombinant adenoviruses were previously demonstrated (FIGS. 9 and 10) to result in activation of the humoral immune response capable of generating anti-CEA IgG antibodies. However, from the splenocyte effector cell pool no significant T-cell activation was measured by interferon spot producing cells in the ELISA SPOT assay. The number of positive spot forming cells in the ADHSP72+CEA-plasmid immunization group is significantly greater than the other groups.

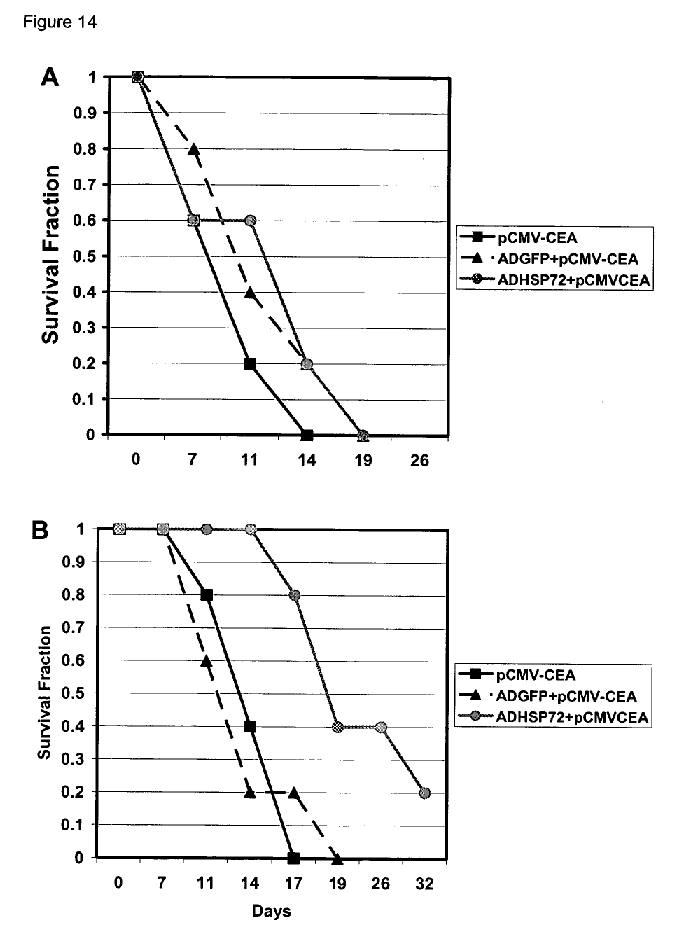

Based on our findings of activation of both humoral and cellular arms of the immune response against CEA, we addressed if vaccination could modify the clinical course of animals with pre-existing tumors. Animals were inoculated with tumor cells and then vaccinated twice at weekly intervals. Survival of animals is show in FIG. 14. All animals formed tumors when challenged with TrampC2 or TrampC2-CEA cells. TrampC2-CEA tumors were slower growing and resulted in death of animals at a later time than TrampC2 challenged animals. However, all animals eventually succumbed to the tumors. Necropsy of animals revealed metastatic disease spread throughout the abdominal cavity. No treatment regimen altered survival of animals given TrampC2 tumors. There was a treatment effect for animals challenged with TrampC2-CEA tumors. CEA-plasmid alone or in the ADGFP+CEA-plasmid vaccination failed to prolong survival. In contrast ADHSP72+CEA-plasmid vaccination prolonged survival by at least 10 days.

### Description of the Figures

FIG. 9 depicts the serum levels of anti-CEA IgG antibody 7 days post vaccination. Animals were vaccinated as described with the regimen represented on the x-axis. Anti-CEA IgG was measured by cellular ELISA. Each value (y-axis) represents the mean and standard deviation for 10 animals performed in triplicate. (*) and (**) denotes comparison groups with statistically significant differences as determined by Student's t-test with p<0.05.

FIG. 10 depicts the serum levels of anti-CEA IgG antibody over time. Animals were vaccinated as described with the regimen represented in the legend. Serum was collected weekly and anti-CEA IgG was measured by cellular ELISA. Each value (y-axis) represents the mean and standard deviation for 10 animals performed in triplicate. (*) denotes statistically significant values (p<0.05) when comparing the pCMV-CEA plus ADHSP72 group to all other groups.

FIG. 11 depicts tumor resistance following immunization. Animals were immunized as described with the regimen represented on the x-axis. Animals were inoculated subcutaneously with 10⁶ cells, one week after the final immunization. Tumor formation was considered positive if animals had at least a palpable tumor assessed one month from the time of tumor cell inoculation. Each value (y-axis) represents percentage of 10 animals. (*) denotes a statistically significant value by chi-square analysis when comparing the AD-HSP72 plus pCMV-CEA group to all other immunization groups and across tumor challenges with either TrampC2-GFP or TrampC2-CEA.

FIG. 12 depicts tumor volumes after challenge of immunized animals. Animals were immunized as described with the regimen represented on the x-axis. Animals were inoculated subcutaneously with 10⁶ cells, one week after the final immunization. Tumors volumes were assessed one month from the time of the initial tumor inoculation. Each value (y-axis) represents average of 10 animals except ADHSP72+pCMV-CEA (n=8).

FIG. 13 depicts ELISA SPOT assays. Pooled splenocytes from 2 animals in each immunization group (x-axis) were used as effector cells against the target cells TrampC2-GFP and TrampC2-CEA. Each value represents the mean of triplicate wells counted for spot forming cells (SFC) per 100,000 pooled splenocytes from 2 animals. (*) represents statistically significant (p<0.05) difference in lysis of TrampC2-CEA target cells in comparison to TrampC2-GFP target cells from the ADHSP72+pCMV-CEA animals. (**) and (***) represents statistically significant (p<0.05) difference in TrampC2-CEA target cell lysis by splenocyte effectors from ADHSP72+pCMV-CEA animals in comparison to target cell lysis from the animals in the ADGFP+pCMV-CEA group and pCMV-CEA alone group, respectively.

FIG. 14 depicts the survival analysis. Survival of animals with pre-existing TrampC2-GFP tumors, (upper panel; A) or pre-existing TrampC2-CEA (lower panel; B) tumors following immunization. Each group consisted of 5 animals. Animals with 25 mm³ or greater tumors were immunized twice 7 days apart with the regimen detailed in the legend. Panel A—Survival analysis by Kaplan-Meier survival statistics did not show differences in survival between immunization groups challenged with TrampC2 cells. Panel B—Survival analysis by Kaplan-Meier survival statistic shows statistically significant prolongation of survival in the ADHSP72+pCMV-CEA immunization group compared to ADGFP+pCMV-CEA or pCMV-CEA alone groups.

### Discussion

Delivery of antigen via plasmid vectors has been shown to elicit humoral and cellular antigen-specific immune responses [13]. Although the duration of responses has varied in different model systems studies, there is potential for generation of long term memory immune responses [18]. DNA vaccines are quite potent at generating high avidity T-cells against specific antigens [19].

We studied CEA antigen in a mouse model to assess the efficacy of a recombinant adenovirus expressing HSP72 to serve as an immune adjuvant to DNA vaccination. CEA is a well characterized tumor antigen with potential as a cancer preventive vaccine [20]. It is a relatively weak immunogen, however. Previously, in a similar mouse model, Song et al. have reported effective CTL memory immune responses with CEA-plasmid vaccination [3]. In their model, at least 4 immunizations were necessary to result in a consistent humoral or cellular immune response. One of the difficulties with DNA vaccination is the requirement of multiple doses to generate protective immunity and in some model systems only weak immune responses can be generated. Consequently, one method to augment immune response generation is by the use of adjuvants [21]. The utilization of cytokines with antigen has been moderately successfully in augmenting both antibody and CTL responses to antigen [22]. In a mouse model, co-immunization with interferon gamma or IL-12 expressing plasmids with CEA-plasmids enhanced T-cell responses [3]. Recombinant virus vectors have also been advantageous for producing strong immune responses, and for increased delivery of antigen to antigen presenting cells. In addition to addressing the adjuvant role of co-immunization of plasmid DNA with recombinant adenovirus, we studied the effects of immunomodulatory HSP72 in a recombinant adenovirus vector to enhance CEA-plasmid vaccination. HSP72 is a potent activator of dendritic cells and can activate other immune effector cells such as NK cells [25]. Furthermore, heat shock proteins deliver maturation signals to dendritic cells by upregulating the expression of co-stimulatory molecules such as B7-1 and B7-2 [26]. HSP72 also has some paracrine ability to induce dendritic cell and T-cell migration [26].

e found that co-injection of recombinant adenovirus vectors with plasmid DNA resulted in a significant increase in anti-CEA IgG production. The increase in IgG levels was 20-40 fold greater than with plasmid DNA vaccination alone after a single immunization. There was not a statistically significant difference in anti-CEA IgG levels between the two groups of CEA-plasmid and recombinant adenovirus administration after a single vaccine administration. However, there is a general trend of increased anti-CEA IgG production over time associated with the ADHSP72. ADHSP72 and CEA-plasmid immunization resulted in significantly higher titers of anti-CEA IgG when assessed at 2, 3 and 4 week intervals which respectively corresponds to the post second, third and fourth immunizations. CEA-plasmid vaccination alone required at least three immunizations before anti-CEA IgG titers are measurable and there is a linear increase in antibody production with subsequent administrations. In numerous other experiments, the immunogenicity of adenovirus has been well documented [27]. The immunogenic nature of recombinant adenoviruses limits their use for corrective gene therapy [28] but may be beneficial in their use as adjuvants.

Interestingly, anti-CEA IgG production was not observed to be a determinant of tumor resistance. Although greater than 30 units of anti-CEA IgG was detected in the CEA-plasmid alone, ADGFP plus CEA-plasmid and ADHSP72 plus CEA-plasmid, only the ADHSP72 plus CEA-plasmid group showed resistance to CEA expressing tumor re-challenge. Animals were inoculated one week after the immunizations were completed with either the TrampC2-GFP or the TrampC2-CEA cell lines. TrampC2-GFP formed tumors in animals of all the treatment groups. In contrast, TrampC2-CEA tumor formation was observed in 100% of animals in the CEA-plasmid alone and the ADGFP plus CEA-plasmid groups but in only 20% of the ADHSP72 plus CEA-plasmid group. 80% of the animals in this group showed tumor resistance which correlated directly with the development of a cellular immune response as shown by T-cell activation against TrampC2-CEA cells by ELISA SPOT assay using splenocytes from these resistant animals. This cellular immune response was CEA specific as IFN producing cells were detected when co-incubated with the TrampC2-GFP cells. Therefore, tumor resistance was conferred by cellular immunity and not humoral immunity. Interestingly, in the 20% of animals in the ADHSP72 plus CEA-plasmid group forming tumors, the tumor size was the same as in the other groups. ELISA SPOT assays using splenocytes from these animals failed to show any T-cell activation against CEA expressing target cells. Therefore, vaccine efficacy could be predicted by the ELISA SPOT assay.

Lastly, we addressed the potential ability of recombinant adenovirus expressing HSP72 to serve as a DNA vaccine adjuvant to modify tumor growth and survival in animals with pre-existing tumors. Animals were inoculated with TrampC2-GFP or TrampC2-CEA cells and when tumors were approximately 25 mm³, animals were immunized twice with ADGFP or ADHSP72 and CEA-plasmid or CEA-plasmid alone. Tumors continued to grow in all animals and there was no difference in tumor progress between groups. TrampC2 is known to be metastatic. Therefore, subcutaneous tumor size is not necessarily an appropriate reflection of tumor burden. As a result, we assessed survival in these animals as well. Animals with TrampC2-GFP tumors in all groups died within 19 days of initial tumor administration. There was no survival benefit to adenovirus administration. TrampC2-CEA bearing animals also died within 19 days in the CEA-plasmid alone group and in the ADGFP plus CEA-plasmid group. In contrast, the ADHSP72 plus CEA-plasmid group showed prolonged survival with one animal showing complete tumor regression. The median time to death was approximately 5 days later than in the CEA-plasmid alone or in the ADGFP plus CEA-plasmid groups.

In addition to assessing the role of recombinant adenoviruses as immune adjuvants for DNA vaccination, we also examined the effects of the immunomodulatory molecule HSP72 to enhance immune response generation. The current paradigm of DNA immunization induced CTL response is through antigen presentation by non-antigen presenting cells which are transfected. We hypothesize that recombinant adenovirus expressing antigens have a similar mechanism for antigen presentation. Co-immunization of plasmid DNA and recombinant adenovirus likely results in some fraction of cells transfected with the plasmid and infected with the adenovirus. HSP72 is immunomodulatory and can bind antigenic peptides of processed antigen as well up-regulating the expression of antigen presenting molecules. Consequently, adenovirus mediated HSP72 expression with plasmid DNA immunization resulted in more efficient antigen presentation and T-cell activation resulting in a cellular immune response capable of conferring tumor immunity and of modifying growth in animals with tumors in our model system. ADHSP72 has potential to be utilized with other antigenic peptides or proteins where generation of cellular immune responses is necessary.

### References Cited in this Example

The following references are cited by number in this example. Applicant reserves the right to challenge the veracity of statements made in these references.
1. Randrianarison-Jewtoukoff, V. and M. Perricaudet, Recombinant adenoviruses as vaccines. Biologicals, 1995. 23(2): p. 145-57.
2. Truckenmiller, M. E. and C. C. Norbury, Viral vectors for inducing CD8(+) T cell responses. Expert Opin Biol Ther, 2004. 4(6): p. 861-8.
3. Song, K., Y. Chang, and G. J. Prud'homme, IL-12 plasmid-enhanced DNA vaccination against carcinoembryonic antigen (CEA) studied in immune-gene knockout mice. Gene Ther, 2000. 7(18): p. 1527-35.
4. Song, K., Y. Chang, and G. J. Prud'homme, Regulation of T-helper-1 versus T-helper-2 activity and enhancement of tumor immunity by combined DNA-based vaccination and nonviral cytokine gene transfer. Gene Ther, 2000. 7(6): p. 481-92.
5. Houry, W. A., Chaperone-assisted protein folding in the cell cytoplasm. Curr Protein Pept Sci, 2001. 2(3): p. 227-44.
6. Haslbeck, M., sHsps and their role in the chaperone network. Cell Mol Life Sci, 2002. 59(10): p. 1649-57.
7. Srivastava, P. K., Heat shock protein-based novel immunotherapies. Drug News Perspect, 2000. 13(9): p. 517-22.
8. Castelli, C., et al., Heat shock proteins: biological functions and clinical application as personalized vaccines for human cancer. Cancer Immunol Immunother, 2004. 53(3): p. 227-33.
9. Srivastava, P. K. and R. J. Amato, Heat shock proteins: the ‘Swiss Army Knife’ vaccines against cancers and infectious agents. Vaccine, 2001. 19(17-19): p. 2590-7.
10. Yedavelli, S. P., et al., Preventive and therapeutic effect of tumor derived heat shock protein, gp96, in an experimental prostate cancer model. Int J Mol Med, 1999. 4(3): p. 243-8.
11. Melcher, A., et al., Tumor immunogenicity is determined by the mechanism of cell death via induction of heat shock protein expression. Nat Med, 1998. 4(5): p. 581-7.
12. Somersan, S., et al., Primary tumor tissue lysates are enriched in heat shock proteins and induce the maturation of human dendritic cells. J Immunol, 2001. 167(9): p. 4844-52.
13. Ada, G. and I. Ramshaw, DNA vaccination. Expert Opin Emerg Drugs, 2003. 8(1): p. 27-35.
14. Okubo, S., et al., Gene transfer of heat-shock protein 70 reduces infarct size in vivo after ischemia/reperfusion in the rabbit heart. Circulation, 2001. 103(6): p. 877-81.
15. Ng, P. and F. L. Graham, Construction of first-generation adenoviral vectors. Methods Mol Med, 2002. 69: p. 389-414.
16. Mittereder, N., K. L. March, and B. C. Trapnell, Evaluation of the concentration and bioactivity of adenovirus vectors for gene therapy. J Virol, 1996. 70(11): p. 7498-509.
17. Conry, R. M., et al., A carcinoembryonic antigen polynucleotide vaccine has in vivo antitumor activity. Gene Ther, 1995. 2(1): p. 59-65.
18. Robinson, H. L., L. A. Hunt, and R. G. Webster, Protection against a lethal influenza virus challenge by immunization with a haemagglutinin-expressing plasmid DNA. Vaccine, 1993. 11(9): p. 957-60.
19. Estcourt, M. J., et al., Prime-boost immunization generates a high frequency, high-avidity CD8(+) cytotoxic T lymphocyte population. Int Immunol, 2002. 14(1): p. 31-7.
20. Thompson, J. and W. Zimmermann, The carcinoembryonic antigen gene family: structure, expression and evolution. Tumour Biol, 1988. 9(2-3): p. 63-83.
21. Sasaki, S., et al., Adjuvant formulations and delivery systems for DNA vaccines. Methods, 2003. 31(3): p. 243-54.
22. Lee, S., et al., Influence of DNA encoding cytokines on systemic and mucosal immunity following genetic vaccination against herpes simplex virus. Microbes Infect, 2003. 5(7): p. 571-8.
23. Chuah, M. K., D. Collen, and T. VandenDriessche, Biosafety of adenoviral vectors. Curr Gene Ther, 2003. 3(6): p. 527-43.
24. Ramshaw, I. A. and A. J. Ramsay, The prime-boost strategy: exciting prospects for improved vaccination. Immunol Today, 2000. 21(4): p. 163-5.
25. Srivastava, P., Roles of heat-shock proteins in innate and adaptive immunity. Nat Rev Immunol, 2002. 2(3): p. 185-94.
26. Li, Z., A. Menoret, and P. Srivastava, Roles of heat-shock proteins in antigen presentation and cross-presentation. Curr Opin Immunol, 2002. 14(1): p. 45-51.
27. Volpers, C. and S. Kochanek, Adenoviral vectors for gene transfer and therapy. J Gene Med, 2004. 6 Suppl 1: p. S164-71.
28. Flotte, T. R. and B. L. Laube, Gene therapy in cystic fibrosis. Chest, 2001. 120(3 Suppl): p. 124S-131S.

Preferred embodiments of the invention are described in the preceding description and examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow.

What is claimed is:

1. A method of treating a disease comprising the steps of (a) contacting a disease cell in a patient with an adjuvant, (b) waiting a therapeutically effective time, and then (c) treating the patient to reduce the disease, wherein further development of disease in the patient is reduced.

2. The method of treating a disease according to claim 1 wherein the antigen is encoded by a polynucleotide and the disease cell is contacted with the polynucleotide, which enters the disease cell, which subsequently produces the antigen.

3. The method of treating a disease according to claim 2 wherein the adjuvant is an immunomodulator.

4. The method of treating a disease according to claim 3 wherein the immunomodulator is a heat shock protein.

5. The method of treating a disease according to claim 4 wherein the heat shock protein is selected from the group consisting of HSP72, HSP 110, HSP/HSC70, gp96(grp96), HSP60, HSP40, HSP90, and BiP (grp78).

6. The method of treating a disease according to claim 3 wherein the immunomodulator is a cytokine.

7. The method of treating a disease according to claim 2 wherein the polynucleotide is contained within the genome of a recombinant adenovirus.

8. The method of treating a disease according to claim 7 wherein the polynucleotide encodes a HSP70 and the recombinant adenovirus is a replication deficient recombinant adenovirus.

9. The method of treating a disease according to claim 1 wherein the therapeutically effective time is a time sufficient to allow for the expression of the antigen in the disease cell.

10. The method of treating a disease according to claim 1 wherein the therapeutically effective time is greater than or equal to 6 hours and less than or equal to 168 hours.

11. The method of treating a disease according to claim 1 wherein the therapeutically effective time is greater than or equal to 6 hours and less than or equal to 72 hours.

12. The method of treating a disease according to claim 1 wherein the therapeutically effective time is greater than or equal to 12 hours and less than or equal to 48 hours.

13. The method of treating a disease according to claim 1 wherein the treating the patient to reduce the disease is selected from the group consisting of surgery, mechanical disruption, ultrasonic disruption, radiation, osmotic shock, application of detergent, application of amphipathic molecules, application of chemotherapy agents, application of complement molecules, application of biological agents, application of immunological agents, application of antibiotics, and application of oncolytic viruses.

14. The method of treating a disease according to claim 1 wherein the disease is cancer.

15. The method of treating a disease according to claim 14 wherein the cancer is selected from the group consisting of colorectal cancer, breast cancer, melanoma and prostate cancer.

16. The method of treating a disease according to claim 1 wherein the disease is an infectious disease.

17. The method of treating a disease according to claim 16 wherein the infectious disease is HIV infection.

18. The method of treating disease according to claim 1 comprising a step of administering a disease antigen to the patient.

19. The method of treating disease according to claim 18 wherein the disease is melanoma, the adjuvant is HSP72 and the antigen is a melanoma-specific tyrosinase.

20. The method of claim 19 wherein the treating the patient to reduce the disease is surgery.

21. A method of treating cancer comprising the steps of:
(a) injecting into a tumor in a patient a composition comprising a recombinant adenovirus, said recombinant adenovirus comprises a polynucleotide encoding an adjuvant polypeptide, wherein said recombinant adenovirus contacts a tumor cell and enters into the tumor cell,
(b) waiting a therapeutic effective time to allow
(i) for the expression of the adjuvant polypeptide in the tumor cell, and
(ii) the adjuvant polypeptide to form a complex with an antigen in the tumor cell, said complex being immunogenic, and
(c) reducing the tumor burden, wherein
(iii) reducing the tumor burden exposes the complex to the host immune system, and
(iv) the patient mounts an immune response to a second tumor cell, and thus preventing further development of a second tumor.

22. The method of treating cancer according to claim 21 wherein the recombinant adenovirus is replication deficient.

23. The method of treating cancer according to claim 21 wherein the adjuvant polypeptide is a heat shock protein selected from the group consisting of HSP72, HSP110, HSP/HSC70, gp96(grp96), HSP60, HSP40, HSP90, and BiP (grp78).

24. The method of treating cancer according to claim 21 wherein the therapeutic effective time is at least 6 hours and no more than 168 hours.

25. The method of treating cancer according to claim 21 wherein the step of reducing the tumor burden is selected from the group consisting of surgery, radiation, chemotherapy, administration of a complement molecule, administration of a biological agent, administration of an immunological agent, administration of an antibiotic, administration of an oncolytic virus, administration of an osmotic shock detergent, and administration of an amphipathic molecule.

26. The method of treating cancer according to claim 21 comprising the step of injecting into the tumor a cancer antigen.

27. The method of treating cancer according to claim 26 wherein the cancer antigen is encoded in a second polynucleotide.

28. A method of treating cancer according to claim 27 wherein the cancer is colorectal cancer and the antigen is carcinoembryonic antigen (“CEA”).

29. A method of treating cancer according to claim 27 wherein the cancer is melanoma and the antigen is melanoma-associated tyrosinase.

30. A method of treating cancer comprising the steps of (a) inducing a tumor cell in a patient to express a heat shock protein, and (b) reducing the tumor burden in the patient, wherein,
(i) the heat shock protein forms a complex with an antigen in the tumor cell,
(ii) the complex is immunogenic, and
(iii) the step of reducing the tumor burden exposes the complex to the host immune system, such that
the patient mounts an immune response to a second tumor cell, thus preventing the further development of a second tumor.

31. The method of treating cancer according to claim 30 wherein the step of inducing a tumor cell in a patient to express a heat shock protein is brought about by application to the tumor of any one of osmotic shock, ultrasound, radiation, chemotherapy, change in temperature, change in pH, pathogen, or biological agent.

32. The method of treating cancer according to claim 30 wherein the step of reducing the tumor burden in the patient is selected from the group consisting of surgery, radiation therapy, chemotherapy, osmotic shock, administration of a complement molecule, administration of a biological agent, administration of an immunological agent, administration of an antibiotic, administration of an oncolytic virus, and administration of an amphipathic molecule.

33. A composition comprising a recombinant adenovirus, which comprises (a) a first polynucleotide encoding a first immunomodulatory agent, and (b) a second polynucleotide encoding a second immunomodulatory agent, wherein the composition is capable of enhancing an immune response in a host vertebrate directed against a disease antigen.

34. The composition according to claim 33 wherein the first immunomodulatory agent is capable of binding to the antigen and presenting the antigen to the host immune system and the second immunomodulatory agent is a cytokine or chemokine.

35. The composition according to claim 34 wherein the first immunomodulatory agent is selected from the group consisting of HSP72, HSP 110, HSP/HSC70, gp96(grp96), HSP60, HSP40, HSP90, and BiP (grp78).

36. A composition according to claim 34 wherein the second immunomodulatory agent is selected from a group consisting of GMCSF, IL 2, IL 12.

37. The composition according to claim 34 wherein the first immunomodulatory agent is HSP72 and the second immunomodulatory agent is GMCSF.

38. The composition according to claim 34, wherein the composition comprises the disease antigen.

39. The composition according to claim 38 wherein the disease antigen is encoded by a polynucleotide.

40. A vaccine composition comprising a vaccine and a recombinant adenovirus comprising a polynucleotide encoding one or more immunomodulatory molecules.

41. The vaccine composition according to claim 40 wherein the immunomodulatory molecule is selected from the group consisting of HSP72, HSP110, HSP/HSC70, gp96(grp96), HSP60, HSP40, HSP90, and BiP (grp78), GMCSF, IL 2, and IL 12.

42. The vaccine composition of claim 40 wherein the polynucleotide encodes a first polypeptide capable of binding an antigen and a second polypeptide that stimulates an immune cell to divide or migrate.

43. A method of treating breast cancer comprising the steps of:
(a) administering a composition to a patient having a tumor burden via intratumor injection, the composition comprising a recombinant adenovirus containing a polynucleotide encoding an immunomodulatory molecule,
(b) waiting a therapeutic effective time, and
(c) reducing the tumor burden, wherein
(i) the immunomodulatory molecule is expressed within a tumor cell, and
(ii) the step of reducing the tumor burden exposes the immunomodulatory molecule and a tumor antigen to the patient immune system, such that
the patient immune system is primed and educated to mount a heightened immune response to a second tumor, thus preventing further development of breast cancer.

44. The method of treating breast cancer according to claim 43 wherein the step of reducing the tumor burden is selected from the group consisting of surgery, radiation, chemotherapy, osmotic shock, administration of a complement molecule, administration of a biological agent, administration of an immunological agent, administration of an antibiotic, administration of an oncolytic virus, and administration of an amphipathic molecule.

45. A method of enhancing the effectiveness of a vaccine comprising the step of injecting into a patient (a) a composition comprising a recombinant adenovirus, said recombinant adenovirus comprising a polynucleotide encoding an immunomodulatory molecule, and (b) a vaccine, wherein (i) the polynucleotide encoding the immunomodulatory molecule is expressed, (ii) the immunomodulatory molecule primes the patient immune system, (iii) the vaccine educates the patient immune system, and (iv) the patient mounts an immune response against a target to which the vaccine is directed, thus enhancing the effectiveness of the vaccine.

46. A method of treating a gastric and duodenal ulcer comprising the step of administrating to a patient (a) a recombinant adenovirus, which comprises a polynucleotide encoding an immunomodulatory molecule and (b) an inactivated Helicobacter pylori bacterium, wherein (i) the polynucleotide encoding the immunomodulatory molecule is expressed in a cell of the patient, (ii) the immunomodulatory molecule primes the immune system, and (iii) the inactivated Helicobacter pylori educates the immune system to the target bacterium Helicobacter pylori, such that the patient mounts an immune response against Helicobacter pylori, said immune response comprises the production of IgA secretory antibodies in the stomach and duodenum.

47. A method of treating AIDs in a patient comprising the step of administrating (a) a recombinant adenovirus, which comprises a polynucleotide encoding an immunomodulatory molecule, and (b) an inactivated HIV, wherein (i) the polynucleotide encoding the immunomodulatory molecule is expressed in a cell of the patient, (ii) the immunomodulatory molecule primes the immune system, and (iii) the inactivated HIV educates the immune system to the target HIV, such that the patient mounts an immune response against HIV.
